(19) United States
(12) Patent Application Publication
(10) Pub. No.: US 2026/0007483 A1
(43) Pub. Date: Jan. 8, 2026

(54) DEVICES, SYSTEMS AND METHODS FOR WEARABLE HAPTIC GUIDANCE AND FEEDBACK

(71) Applicant: Board of Regents, The University of Texas System, Austin, TX (US)

(72) Inventors: Ann Majewicz Fey, Austin, TX (US); Bharat Mathur, Austin, TX (US); Quentin Cole Schuelke, Lockhart, TX (US); Roman Mykhailyshyn, Austin, TX (US)

(21) Appl. No.: 19/258,545

(22) Filed: Jul. 2, 2025

Related U.S. Application Data
(60) Provisional application No. 63/667,361, filed on Jul. 3, 2024.

(51) Int. Cl. A61B 34/00; A61B 34/35; B25J 13/02; B25J 13/08

(52) U.S. Cl. CPC A61B 34/76; A61B 34/35; A61B 2034/741; B25J 13/025; B25J 13/084

(57) ABSTRACT

A wearable haptic guidance and feedback device comprises a sleeve, a grounding mechanism attached to the sleeve, at least one actuator attached to the grounding mechanism, and at least one actuation mechanism mechanically coupling the at least one actuator to at least one of the sleeve and the grounding mechanism. Related systems and methods are also disclosed.

### CROSS-REFERENCE TO RELATED APPLICATIONS

This application claims priority to U.S. provisional application No. 63/667,361 filed on Jul. 3, 2024, incorporated herein by reference in its entirety.

### STATEMENT REGARDING FEDERALLY SPONSORED RESEARCH OR DEVELOPMENT

This invention was made with government support under CMMI2102250 awarded by the National Science Foundation. The government has certain rights in the invention.

### BACKGROUND OF THE INVENTION

Robotic-assisted surgery is now well-established in clinical practice and has become the gold-standard clinical treatment option for several clinical indications [see C. D'Ettorre, et al., 2021]. As computer-aided surgery becomes increasingly prevalent, human-machine interaction is a key factor for the success of autonomy in surgical robotics [see A. Attanasio, et al., 2021].

The use of haptics for human-machine interaction in surgical robotic systems has been widely explored both in research and clinically. In clinical practice, haptic interaction is used for bimanual teleoperation in systems like Senhance (Asensus Surgical, North Carolina, USA) and REVO-I (Revo Surgical, Seoul, Korea) [see P. Culmer, et al. 2020]. In cooperatively controlled systems such as the Mako RIO (Stryker Inc., Michigan, USA) and Acrobot [see J, Cobb, et al., 2006], haptics is used to prescribe active constraints to the patient's anatomy and the robot prohibits movement of the surgical tool beyond this boundary [see C. J. Payne, et al., 2014]. While these mechanically grounded, teleoperated, and cooperatively controlled robotic platforms have seen both commercial and clinical success, uptake of these robots remains moderate because of their high cost, large physical footprint, and long setup times.

Haptic systems are often classified as grounded or ungrounded. Grounded haptic devices are physically fixed to external structures and can deliver high-fidelity, multi Degree-of-Freedom (DoF) force feedback. Their mechanical stability enables precise force rendering required during complex or delicate interactions. However, these systems inherently limit user mobility and constrain the interaction workspace. In contrast, ungrounded haptic devices prioritize wearability and portability. By eliminating the need for a fixed base, these systems enable users to move naturally and operate in more immersive or dynamic environments. Yet, without external grounding, they struggle to replicate the force magnitude and feedback fidelity achieved by grounded systems. This tradeoff highlights the need for novel haptic interfaces that preserve fidelity while maintaining flexibility.

Small footprint, lower cost surgical systems such as the commercially available NAVIO (Smith & Nephew, Hertfordshire, UK) and TMINI (THINK Surgical, California, USA), or the in-research systems such as Craniostar [see G. J. Kane, et al., 2009] and other handheld robots for orthopedic and laparoscopic applications [see S. S. Hung, et al., 2021; F. Focacci, et al., 2007; J. Shang, et al., 2011; R. J. Hendrick, et al., 2014] may define the next generation of surgical robotic systems. These handheld robots integrate sensing and onboard actuation to provide specific assistance such as active guidance, force control, and tremor suppression to the surgeon [see C. J. Payne, et al., 2014]. They combine the benefits of direct human operation for gross movements with the precision of robotic control for fine motions [see X. Lou, et al., 2023]. Though advantageous in low-resource settings and better off than manual surgery or navigated surgical systems, the assistance offered is limited due to their small actuated workspaces.

Diverse ungrounded haptic interfaces have been developed using various haptic modalities and actuation methods. Despite the extensive research, most devices focus on unimodal (tactile or kinesthetic) actuation strategies, often lacking comprehensive guidance in all six degrees of freedom (DoFs) of the human arm.

Haptic feedback in wearable systems has traditionally relied on vibrotactile actuation, which uses localized vibrations to convey tactile cues. These systems are widely adopted due to their simplicity, low cost, small size, and fast response. Vibrotactile feedback is particularly effective for delivering alert-based cues, such as notifications or directional prompts in constrained tasks like navigation. As a result, vibration motors are a popular choice in wearable devices. However, vibrotactile actuation has significant limitations in delivering proportional or continuous directional feedback. Vibrations are inherently one-dimensional and spatially diffused, which restricts their ability to convey nuanced proprioceptive information. The large receptive fields of deep mechanoreceptors responsible for vibration detection (i.e., Pacinian corpuscles) require wide spacing between actuators to avoid interference. Additionally, vibrotactile feedback is prone to desensitization over prolonged or repeated use.

Skin stretch feedback, on the other hand, has emerged as a powerful method for delivering continuous and more intuitive multidimensional proprioceptive cues. By applying controlled shear forces to the skin, these systems stimulate superficial, slow-adapting mechanoreceptors—avoiding the limitations associated with deeper Pacinian corpuscles. This enables higher spatial resolution, as actuators can be placed closer together without interference. Unlike vibrotactile feedback, which often requires multiple actuators to create a sensation of motion through sequential activation, skin stretch can inherently convey directionality—such as left versus right—with fewer components, simplifying device design. Another key advantage of skin stretch is its resistance to desensitization. This makes it particularly suited for long-duration tasks such as immersive VR/AR interactions, surgical training, and teleoperation. Moreover, skin stretch offers a more natural perception of movement by simulating shear forces rather than normal forces, enhancing realism and comfort. Skin stretch devices have been implemented using various actuation strategies, including bands, rockers, or hybrid mechanisms. These configurations allow for directional skin stretch in longitudinal, latitudinal, circumferential, or rotational patterns across the body. However, all prior devices focus on a single axis or a limited subset of motion directions, and none provide haptic guidance across all six degrees of freedom. SCWEES (see A. Haynes, M. F. Simons, T. Helps, Y. Nakamura, and J. Rossiter, “A wearable skin-stretching tactile interface for human-robot and human-human communication,” IEEE Robotics and Automation Letters, vol. 4, no. 2, pp. 1641-1646, 2019) is a device that uses Shape Memory Alloy (SMA) actuation to deliver lateral skin stretch, effectively replicating the extension and contraction sensations at the wrist joint. Chen et al. (see C.-Y. Chen, Y.-Y. Chen, Y.-J. Chung, and N.-H. Yu, “Motion guidance sleeve: Guiding the forearm rotation through external artificial muscles, “in Proceedings of the 2016 CHI Conference on Human Factors in Computing Systems, ser. CHI '16. New York, NY, USA: Association for Computing Machinery, 2016, p. 3272-3276) developed a wearable sleeve actuated by artificial muscles to guide forearm pronation and supination. Similarly, Kayhan et al. (see O. Kayhan, A. K. Nennioglu, and E. Samur, “A skin stretch tactor for sensory substitution of wrist proprioception,” in 2018 IEEE Haptics Symposium (HAPTICS), 2018, pp. 26-31) introduced a servo motor-driven skin stretch interface capable of providing haptic feedback for three-degree-of-freedom wrist movements. For translations of the forearm, the work by Kuniyasu et al. (see Y. Kuniyasu, M. Sato, S. Fukushima, and H. Kajimoto, “Transmission of forearm motion by tangential deformation of the skin,” in Proceedings of the 3rd Augmented Human International Conference, ser. AH '12. New York, NY, USA: Association for Computing Machinery, 2012) uses tangential skin deformation for up-down and left-right translations.

Fluidic actuation allows for the relocation of electromechanical components off-board, resulting in a more lightweight, compact, and streamlined haptic interface. Studies [see M. Raitor, et al., 2017; B, Jumet, et al., 2023] have introduced wearable devices equipped with pneumatic pouches, capable of active inflation/deflation to deliver haptic cues in 2 and 3 directions, respectively. Furthermore, Luo [see x. Luo, et al., 2023] presents pneumatic pouches integrated into the handle of a handheld robot, offering 3 Degrees of Freedom (DoF) orientation guidance.

Kinesthetic haptic feedback provides users with real forces along their joints, guiding their movements. In the context of upper limb guidance, kinesthetic feedback has mostly been explored for 2 DoF wrist motion. Notable implementations include MRI-compatible ultrasonic motors [see E. H. Skorina, et al., 2018], and utilizing pneumatic artificial muscles to provide precise kinesthetic guidance to the wrist joint [see A. Erwin, et al., 2017]. Margineanu [see D. Margineanu, et al., 2018] developed an arm exoskeleton for teleoperation, offering haptic feedback across 5 arm joints, excluding radial and ulnar deviation of the wrist. Controlled orientation gyroscopes can also be used to produce ungrounded kinesthetic feedback. This has been explored in handheld devices [see J. M. Walker, et al., 2018; K. N. Winfree, et al., 2009].

Thus there is a need in the art for improved devices, systems, and methods of wearable haptic guidance and feedback.

### SUMMARY OF THE INVENTION

Some embodiments of the invention disclosed herein are set forth below, and any combination of these embodiments (or portions thereof) may be made to define another embodiment.

In one aspect, a wearable haptic guidance and feedback device, comprises a sleeve, a flexible grounding mechanism attached to the sleeve, configured to conform to a wearer's anatomy and distribute a reaction force to said anatomy, at least one actuator attached to the grounding mechanism, and at least one actuation mechanism mechanically coupling the at least one actuator to at least one of the sleeve and the grounding mechanism.

In one embodiment, the device is ungrounded.

In one embodiment, the device further comprises one or more sensors.

In one embodiment, the one or more sensors comprise at least one of a flexible force sensor, an inertial measurement unit, a force sensor, a position sensor, a velocity sensor, an acceleration sensor, an orientation sensor, a flex sensor, and an encoder.

In one embodiment, the sleeve comprises at least two layers.

In one embodiment, wiring for the at least one actuator or sensor is between the layers of the sleeve.

In one embodiment, the grounding mechanism comprises chain mail.

In one embodiment, the chain mail comprises hexagons.

In one embodiment, the grounding mechanism is attached to the sleeve proximate to a bicep location and a forearm location.

In one embodiment, the grounding mechanism comprises a thread passing through at least a portion of the grounding mechanism, and a fixation mechanism configured to tighten the thread and grounding mechanism. The fixation mechanism can comprise any suitable mechanism including a ratcheting mechanism, elastic, and/or Velcro.

In one embodiment, the at least one actuator comprises eight actuators.

In one embodiment, two of the eight actuators are positioned in a first location of the grounding mechanism, and six of the eight actuators are positioned in a second location of the grounding mechanism.

In one embodiment, the first location is proximate to a forearm and the second location is proximate to a bicep.

In one embodiment, the at least one actuator comprises at least one of a servo motor, a brushed DC motor, a brushless DC motor, a linear actuator, an electroactive polymer, a shape memory alloy, and a pneumatic actuator.

In one embodiment, the at least one actuation mechanism comprises linear and tendon-driven actuation mechanisms.

In one embodiment, the linear actuation mechanisms comprise rack and pinion mechanisms operating flexible rods, a ball screw, a lead screw, or a pneumatic, and wherein the tendon-driven actuation mechanisms comprise a tendon comprising a cord.

In one embodiment, the device further comprises actuated anchoring points interfacing with at least one of the rods or tendons.

In one embodiment, the rods and tendons comprise a plastic or a metal.

In another aspect, a wearable haptic guidance and feedback system comprises the wearable haptic guidance and feedback device as described above, and a computing system communicatively connected to the wearable haptic guidance and feedback device.

In one embodiment, the computing system comprises a processor and a non-transitory computer-readable medium with instructions stored thereon, which when executed by the processor, perform steps comprising actuating the at least one actuator.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 depicts an exemplary wearable haptic guidance and feedback device, also known as an Intuitive Six DoF Remote Haptic Assistance Device (ItS-RHAD), in accordance with some embodiments.

FIG. 2 compares grounded and handheld surgical systems with wearable haptic guidance features in accordance with some embodiments.

FIG. 3 details exemplary 3D printed components of the haptic device in accordance with some embodiments.

FIG. 4 depicts actuation methods for each direction of motion in accordance with some embodiments.

FIG. 5 depicts details of an experimental setup in accordance with some embodiments.

FIG. 6 is a plot depicting average response time, standard deviation, and outliers in a correlation test using a Lambda.7 haptic device in accordance with some embodiments.

FIG. 7 is a plot depicting results of a correlation test in free space in accordance with some embodiments.

FIG. 8 is a plot depicting results of a correlation test with Force Dimensions Lambda.7 in accordance with some embodiments.

FIG. 9 depicts exemplary compound tasks in accordance with some embodiments.

FIGS. 10A-10D depict exemplary XZ drawings task results in accordance with some embodiments.

FIGS. 11A-11C depict exemplary XY drawings task results in accordance with some embodiments.

FIGS. 12A-12C depict exemplary data over time representations for various tasks in accordance with some embodiments.

FIGS. 13A-13B depict combined metrics for drawing and compound tasks in accordance with some embodiments.

FIG. 14 depicts an exemplary computing environment in which aspects of the invention may be practiced in accordance with some embodiments.

### DETAILED DESCRIPTION OF THE INVENTION

It is to be understood that the figures and descriptions of the present invention have been simplified to illustrate elements that are relevant for a clearer comprehension of the present invention, while eliminating, for the purpose of clarity, many other elements found in devices, systems and methods of wearable haptic guidance and feedback. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20%, +10%, +5%, +1%, and +0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein are devices, systems and methods of wearable haptic guidance and feedback.

Disclosed herein are innovative techniques to provide ungrounded haptic feedback. The methods were tested to create an exemplary device named “ItS-RHAD”, a novel wearable interface that provides high-fidelity 6 degree of freedom (DoF) ungrounded proprioceptive haptic guidance. The interface combines kinesthetic guidance and tendon-driven skin stretch mechanisms and introduces a novel haptic delivery technique called “Referred Torques Mediated by Skin Stretch.” The design and fabrication of ItS-RHAD are presented below, along with rudimentary control strategies that were used to conduct a basic functional validation. Participants were asked to identify the direction in which they were provided guidance. In a series of experiments, the haptic interface guided participants through various real-world tasks that required a sequence of translation and orientation movements. The forces rendered by the device are highly intuitive, and wearers correctly identified guidance in 89% of cases. The interface also successfully guided wearers with high precision, with sub-centimeter precision in translations and within 5 degrees in orientations. This demonstrates the early feasibility of the interface in providing precise proprioceptive guidance, which can be used to train users to perform surgical tasks or guide them to manipulate navigated hand-held surgical tools.

In some embodiments, each degree of freedom is actuated independently, and via independent actuation mechanisms.

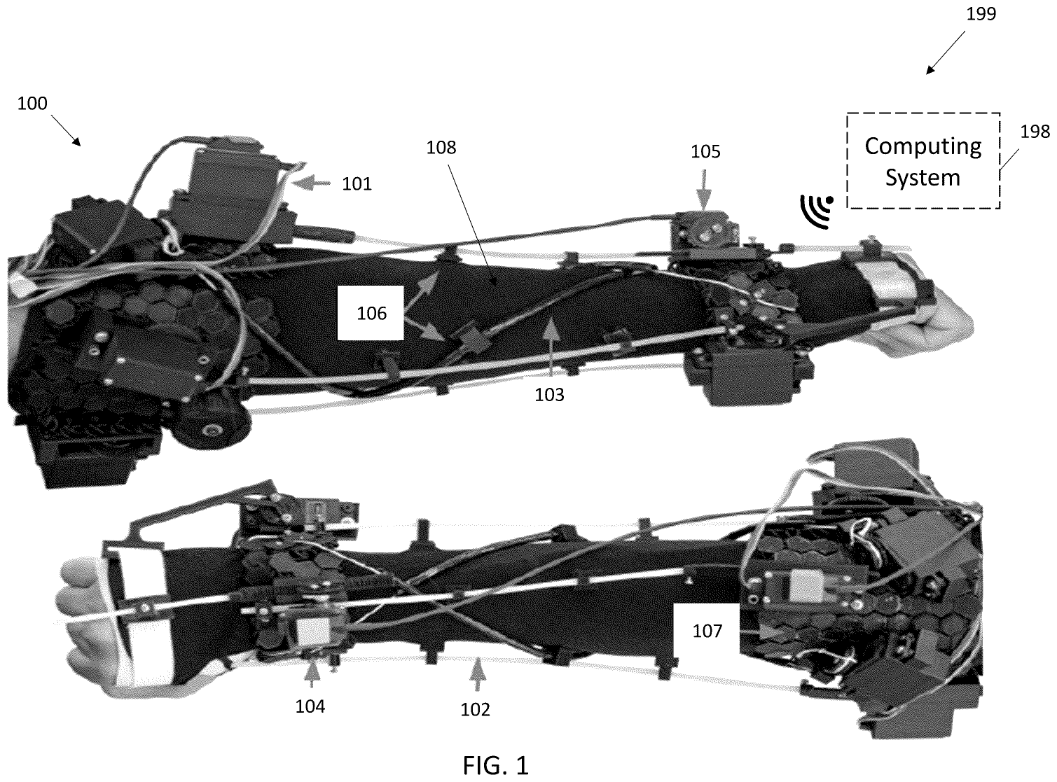

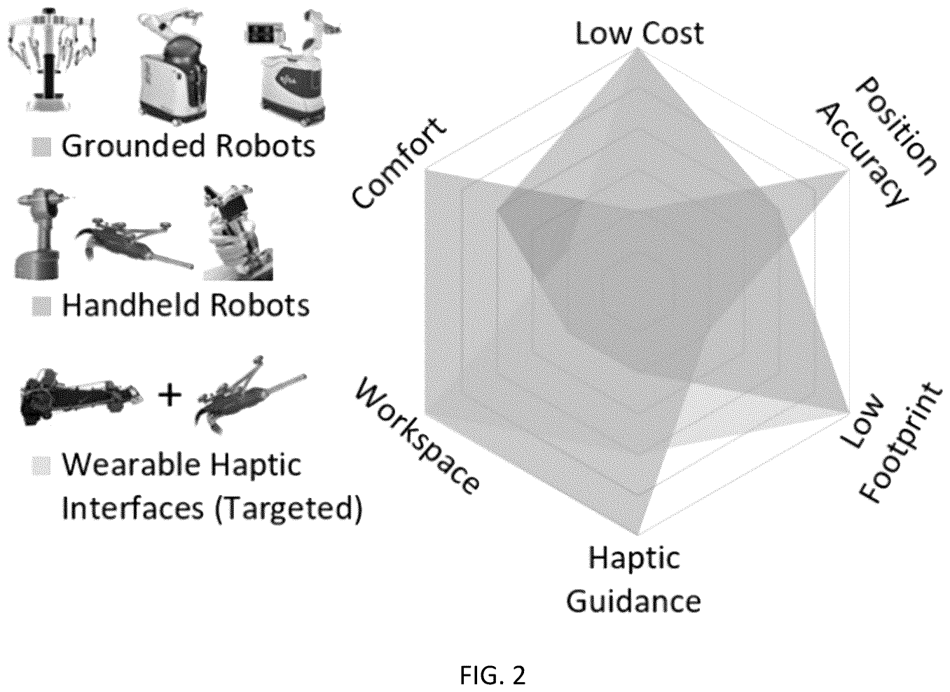

To enhance the potential of navigated surgical instruments by expanding their workspace, disclosed is a wearable haptic device, such as the one illustrated in FIG. 1, that can provide ungrounded haptic guidance. Such an interface can provide effective, low-cost robot-assisted surgical solutions. The advantages of such a system are hypothesized in FIG. 2. Based on similar principles, Luo [see X. Lou, et al., 2023] presented a haptic interface to guide a hand-held concentric tube robot. In addition to integration with existing navigated handheld devices, prior work [see L. R. Reyes, et al., 2022; S. Basu, et al., 2016] has shown the feasibility of wearable haptic interfaces in surgical training for complex skill acquisition and telementoring for surgery. Rossa [see C. Rossa, et al., 2016] showed the feasibility of a haptic wristband that could be used to provide ultrasound-assisted guidance in needle steering maneuvers.

Disclosed herein are new techniques that enable precise 6 degree-of-freedom haptic guidance by combining kinesthetic and tactile sensations. This is achieved through a unique haptic delivery method called “Referred Torques Mediated by Skin Stretch.” To evaluate the effectiveness of these techniques, an experimental wearable prototype was developed named “ItS-RHAD” (Intuitive Six DoF Remote Haptic Assistance Device). It is worth noting that ItS-RHAD is the first wearable, ungrounded haptic device that can guide all six degrees of freedom, including hand translation (x, y, z), as well as wrist orientation (roll, pitch, yaw), enabled by attachment above the elbow.

Grounded haptics involve haptic devices that are physically anchored to an external structure, providing a stable reference point for delivering force feedback. These devices can exert larger forces and more precise control since they are attached to a stationary frame or base. Ungrounded haptics involve wearable haptic devices that are not anchored to an external structure. Instead, they are typically attached directly to the user's body, such as the hands, arms, or fingers, providing a more mobile and immersive experience.

The device was designed with the goal of exploring a haptic interface with three core objectives. First, the haptic interface should provide ungrounded guidance cues that closely resemble externally grounded haptic guidance. Second, the guidance signals should be situated within the user's task space and prioritize maximum intuitiveness. Third, the interface should cause minimal obstruction to the user's natural movements while performing the task.

Referring now to FIGS. 1-4, an exemplary wearable haptic guidance and feedback device 100 and system 199 are shown.

In some embodiments, device 100 comprises a wearable haptic guidance and feedback device including a sleeve 108, a grounding mechanism 107 attached to the sleeve 108, at least one actuator (101, 104) attached to the grounding mechanism 107, and at least one actuation mechanism (102, 103) mechanically coupling the at least one actuator (101, 104) to at least one of the sleeve 108 or the grounding mechanism 107. In some embodiments, the at least one actuation mechanism (102, 103) mechanically couples the at least one actuator (101, 104) positioned proximate to a first end of the device 100 to a location of at least one of the sleeve 108 or the grounding mechanism 107 at a second end of the device 100. In some embodiments, the device is ungrounded in relation to the ground. In some embodiments, the grounding mechanism 107 comprises a rigid but flexible surface such that it can conform to the curvature of the arm or other body part, and distribute forces over a large surface area.

In some embodiments, the device 100 further includes one or more sensors. In some embodiments, the one or more sensors comprise at least one of a flexible force sensor, an inertial measurement unit, a force sensor, a position sensor, a velocity sensor, an acceleration sensor, an orientation sensor, a flex sensor, and an encoder, an EMG sensor, an external electromagnetic, RGB, or infrared based position tracking system with fiducials (i.e. NDI, Optitrack®, Vicon®), or other suitable sensors or combinations thereof. The one or more sensors can be placed in any suitable location of the device 100. In some embodiments, the one or more sensors are configured to measure position and/or orientation of the wrist and/or elbow joints.

In some embodiments, the sleeve 108 is double layered. In some embodiments, wiring for the at least one actuator (101, 104) is positioned at least partially between the layers of the sleeve 108.

In some embodiments, the grounding mechanism 107 comprises 3D printed chain mail. In some embodiments, the chain mail comprises hexagons or any other suitable shapes. In some embodiments, the grounding mechanism 107 is attached to the sleeve 108 proximate to first and second ends. In some embodiments, the first and second ends comprise a bicep location and a forearm location. In some embodiments, the grounding mechanism 107 comprises a fastening method 109 such as thread or cord passing through each portion of the grounding mechanism and a tightening mechanism configured to tighten the thread or cord thus tightening the grounding mechanism 107.

In some embodiments, the at least one actuator (101, 104) comprises eight actuators. In some embodiments, two of the eight actuators (101, 104) are positioned in a first location of the grounding mechanism, and six of the eight actuators (101, 104) are positioned in a second location of the grounding mechanism 107. The actuators-actuated locations can be any locations on the body that have desired relative motion between. In the case of skin-stretch, the actuator-actuated devices can also be on the same body part. It is not necessary for the actuator-actuated locations to span a joint. In some embodiments, the first location is configured to be placed on or proximate to a forearm and the second location is configured to be placed on or proximate to a bicep. In some embodiments, the at least one actuator comprises at least one of a servo motor, a brushed DC motor, a brushless DC motor, a linear actuator, a pneumatic actuator, a shape memory alloy, a piezoelectric, a hydraulic, an electrostatic, a magnetic, an electroactive polymer, a microfluidic, a carbon nanotube muscle, a dielectric nanotube, or any other suitable actuator or combinations thereof.

In some embodiments, the at least one actuation mechanism (102, 103) comprises linear and tendon-driven actuation mechanisms. In some embodiments, the linear actuation mechanisms comprise rack and pinion mechanisms 105 operating flexible rods 102, and the tendon-driven actuation mechanisms comprise a tendon 103 comprising a cord where the tendon is wound around a spool driven by a motor. In some embodiments, the device 100 further comprises actuated anchoring points (nodes) 106 interfacing with at least one of the rods 102 or tendons 103. In some embodiments, the rods 102 and tendons 103 comprise nylon, a plastic, a metal, for example aluminum, titanium, nitinol, a composite material, a plant fiber, spider silk, Kevlar, an elastomer, silicon rubber, or other suitable materials, or combinations thereof. Any rigid or flexible material can be used. Suitable materials for the rods should have sufficient rigidity to transmit a compressive and/or extension force, while maintaining enough flexibility to minimize impediment to user range of motion. Tendons can be any material that forms to the arm and has the strength to transmit tension forces. In some embodiments, the device 100 includes a computing system, a transceiver for wired and/or wireless communication, and/or a wired or standalone (battery) power source.

In some embodiments, the rods 102 are rigid enough to transfer compression forces. In some embodiments, buckling is prevented by the placement of intermediate nodes along the length of the arm. In some embodiments, the tendons 103 are flexible enough to wrap around the arm and strong enough to handle the tension when pulling.

In some embodiments, a wearable haptic guidance and feedback system 199 comprises the wearable haptic guidance and feedback device 100, and a computing system 198 communicatively connected to the wearable haptic guidance and feedback device 100. In some embodiments, the computing system 198 comprises a processor and a non-transitory computer-readable medium with instructions stored thereon, which when executed by the processor, perform steps such as actuating the at least one actuator, receiving and analyzing sensor data to determine a flexible force, an inertial measurement, a force, a position, a velocity, an acceleration, and/or an orientation of the device 100 or components of the device (101, 102, 103, 014, 105).

FIG. 1 shows exemplary device 100 and system 199 being worn. In some embodiments the device 100 is an intuitive six degree of freedom remote haptic assistance device (ItS-RHAD) which includes actuators (101, 104), flexible rods 102, tendons 103 in a spiral wrap, a rack and pinion 105 actuation mechanism, nodes 106, and 3D printed chain mail 107 attached to a sleeve 108.

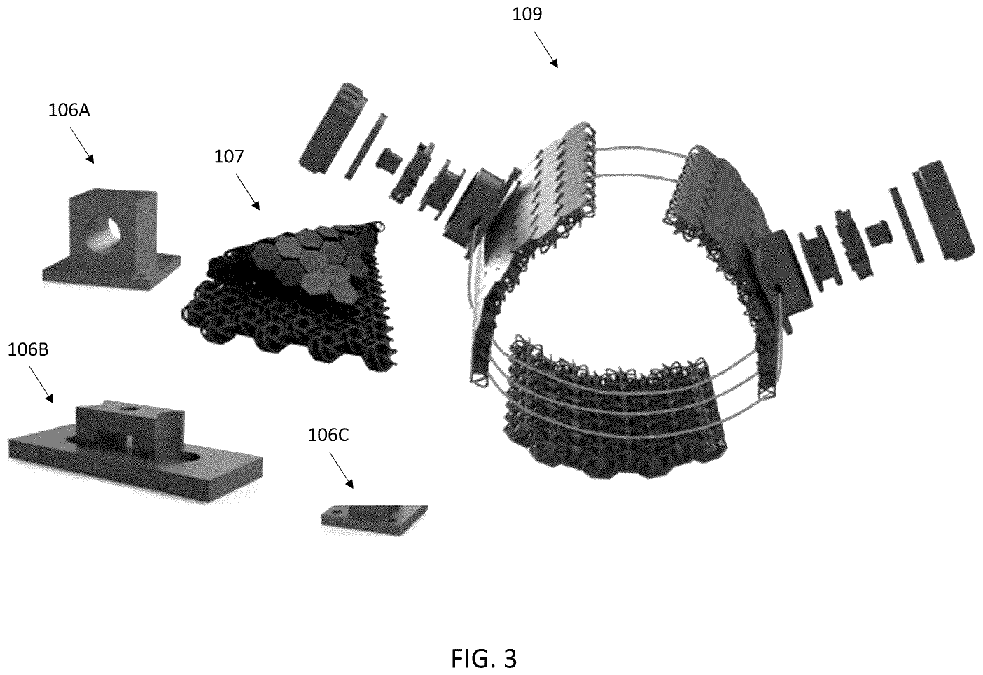

FIG. 3 shows exemplary 3D printed components of the device 100 including a tendon node 106A, flexible 3D printed chain mail 107, a forearm attachment node 106B, a nylon rod node 106C, and a fastening mechanism for the chain mail 109 comprising ratchets.

In one embodiment, the haptic interface by may be created by sewing 3D-printed parts onto an elastic arm sleeve, such as one used by cyclists. This helps to achieve the aim of keeping the wearer's hands free to perform tasks without obstruction.

In some embodiments, the haptic interface can be configured for use on and/or across any suitable body locations including, but not limited to, fingers, hands, wrists, forearms, elbows, upper arms, shoulders, chest, back, stomach, hips, upper legs, knees, lower legs, ankles, feet, toes, neck, and/or jaw.

In some embodiments, the system can be adapted for an elbow joint. The existing implementation of the haptic methods on the arm could be modified in a few ways. For example, using tethers in place of the semi-rigid rods on the inside and outside would allow for greater range of motion in the elbow. By eliminating the rods along the axis in which the elbow naturally bends, one can increase comfort and range of motion of the user while still retaining two rigid rods which could be used to give the translate forward and backward command. If further flexibility was desired, all rods could be replaced with tethers. In some embodiments, in order to retain the full suite of commands a noticeable pre-tension in the tethers can be used so that a release of that tension could be associated with the translate forward motion, since this design would eliminate any ability to push the wrist away from the anchors on the bicep. However this scheme is still feasible given previous results suggesting that relative pressure is more important in the interpretation of commands than absolute pressure. Additionally, the rack and pinion system used for pitch of the wrist can be replaced with two tethers anchored on top and bottom of the hand that operated in a scheme similar to the one described above.

In some embodiments, the system can be adapted for an ankle joint. In a manner similar (kinesthetic feedback) to the pitch or yaw actuator of the above-described sleeve, pivot joints can be applied above the ankles to cue extension and flexion of the ankle joint. Another motor and pivot joint can be added orthogonally if two degrees of freedom were required or desired.

In some embodiments, the system can be adapted for a knee joint. Control of the knee can be achieved using a similar tether scheme or with semi-rigid rods. The rods allow for an extension signal to be provided and would provide the most intuitive control. However, some applications may require a large range of motion in which case tethers can be used for their flexibility. With motors anchored on the thigh, both front and back, the pull element of the push-pull mechanism used to command up-down or left-right in the above-described sleeve can be used to induce motion in the knee. For simple extension flexion, one motor on the front and back would suffice, however two motors on each the front and back can offer more stabilizing control. A contraction of all motors can correspond to a lifting of the knee and the release of that tension can correspond to relaxation of the knee to its natural position, since any extension of the knee must be accompanied by a prior flexion. In the case of the semi-rigid rods, the user would feel an active cue to extend rather than simply the contrast of release.

In some embodiments, the system can be adapted for a hip joint. In order to guide users' hip motion, and that of the entire leg as detailed below, an orthopedic belt-like device can be used for anchoring three motors to perform similar functions as those described above. Two of the motors can be connected to tethers anchored on either the front or back side of the thigh, while the third can be connected to a semi-rigid rod running down the outside of the leg. The tethers on the front and back would be responsible for the raising and lowering of the leg, while the rod, when contracted would instruct an abduction or when extended and adduction of the leg.

In some embodiments, the system can be adapted for a full leg. The case of the full leg (hip, knee, ankle) may be regarded as analogous to the original use case (shoulder, elbow, wrist). Further, multiple devices may work in tandem (i.e. hip, knee, and/or ankle, or shoulder, elbow, and/or wrist) to provide further haptic feedback. The multiple devices can be communicatively connected to each other via any suitable wired or wireless means.

In some embodiments, the system can be adapted for a torso. For guidance of the torso and the relative motion between upper and lower body, a scheme similar to the rotation of the arm could be employed. An orthopedic belt or corset can be used to anchor motors on each side of the waist. One motor on each side would be connected to a tether which would wrap around the torso in a helical manner and be anchored on the upper torso near the shoulders. Contraction of these tethers would lead to the sensation of twisting, just as it did in the arm. Another set of motors can be connected to tethers that ran vertically up the sides of the user and anchored under the armpits. Tension in these tethers would induce a lean to the tightened side. And finally, a set of motors placed on the front and back of the torso are able to signal the user to bend forward or backward.

In some embodiments, to increase comfort, two sleeves can be sewn together to create a dual-layered sleeve. In some embodiments, wiring for the actuators runs between the two layers to minimize interference.

In some embodiments, the ItS-RHAD system comprises four main components: force grounding, actuation, end-effectors, and nodes.

In some embodiments, to achieve the objective of generating high-fidelity ungrounded forces an efficient body grounding mechanism is needed. In one embodiment the solution is a flexible 3D printed chain-mail, comprising hexagons, which is placed on both the bicep and forearm (shown in FIG. 1). In some embodiments, the chain-mail comprises 50 to 100 hexagons, 60 to 70 hexagons, 64 hexagons, or any suitable number of hexagons. In some embodiments, the hexagons have a side length of 5 mm to 25 mm, 10 mm to 15 mm, 12 mm, or any other suitable length. Furthermore, the chain-mail may comprise any suitable shapes or combinations thereof, including but not limited to, circles, triangles, quadrangles, pentagons, hexagons, octagons, or similar.

This chain-mail is inspired by 3D printed fabrics designed for space applications [see U.S. Pat. No. 11,077,655, issued on Aug. 3, 2021, and incorporated herein by reference]. It provides a large, rigid surface that can distribute reaction force effectively while being flexible enough to fit the user's arm shape, improving ergonomics. Since the mesh rests on soft tissue, rigid placement of the actuators is challenging for effective grounding. To ensure improved rigidity, a nylon thread of diameter 0.2 mm to 0.6 mm is fed through each mesh and tightened by a ratchet mechanism to provide a snug fit around the arm.

In some embodiments, an Arduino Mega 2560 was used as the main microcontroller, which communicates with a host computer (Intel Core i7-11800H @ 2.30 GHZ, 16 GB RAM) using ROSSerial. The host computer runs high-level experimental tasks and transmits normalized control inputs in the range of [−1, 1] to the Arduino via ROS custom messages at 1,000,000 bits/second. The Arduino then converts these commands into PWM signals to drive the servo motors.

In some embodiments, for actuation seven MG996R motors and one SG90 servo motor, all with a 180° range, are utilized. Depending on device configuration, motors should simply provide enough force to manipulate the actuated devices. It should be noted that given a motor with a specific range of motion, speed, and torque the characteristics of the motor could be modified by gearing or other transmission devices. As seen in FIG. 1, two of these motors are mounted on the forearm mesh while the remaining six are mounted on the bicep mesh. In some embodiments, custom-designed low-profile mounts are used to attach the motors to the meshes. As the entire interface is soft and flexible, it expands and contracts with the wearer. Further the placement of motors avoids inter-actuator collision in users with smaller arms.

Any suitable motors can be utilized including, but not limited to, servo motors, brushed DC motors, brushless DC motors, linear actuators, and pneumatic actuators. In some embodiments, one or more of the motors are associated with a corresponding one or more sensors for measuring at least one of force, position, velocity, acceleration, and orientation. In some embodiments, these one or more sensors comprise a flexible force sensor, an inertial measurement unit, a force sensor, a position sensor, a velocity sensor, an acceleration sensor, an orientation sensor, a flex sensor, and an encoder, an EMG sensor, an external electromagnetic, RGB, or infrared based position tracking system with fiducials (i.e. NDI, Optitrack®, Vicon®), or other suitable sensors or combinations thereof.

In some embodiments, the force rendering system employs two types of actuation mechanisms-linear and tendon-driven. To generate linear forces, integrated rack and pinion mechanisms are integrated into the motor mounts (see FIG. 1) which operate flexible rods. In some embodiments, the flexible rods have a diameter of 1 mm to 10 mm, 2 to 5 mm, about 3 mm, or any other sizes that offer suitable flexibility and strength. In some embodiments, the flexible rods comprise nylon or other suitable materials. These rods are rigid enough to transmit tension and compression forces, yet compliant enough to enable free movement of the arm without resistance from the haptic feedback. On the other hand, for tendon-driven axes, cord is used as a flexible tendon. In some embodiments, the cord has a diameter of 0.5 mm to 1 mm, or 0.7 mm to 0.8 mm, about 0.75 mm, or any other sizes that offer suitable flexibility and strength. In some embodiments the cord comprises nylon fishing braid, nylon monofilament, or other suitable materials. The motor mounts for these axes feature a small pillar with a fixed aperture, which ensures that the tendon is always routed into the spool consistently.

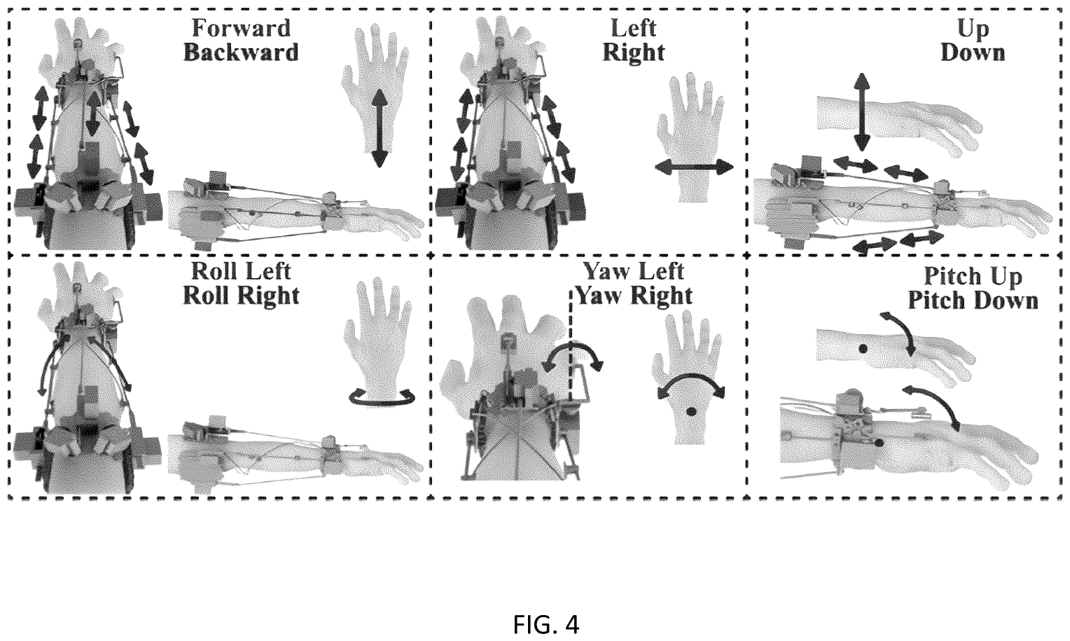

FIG. 4 illustrates the actuators and passive components that are employed to provide haptic feedback in every axis. In this figure, elements corresponding to the active axis are highlighted, while others are grayed out for clarity. Color-coded arrows indicate the direction of actuation of the tendons and rods for each degree of freedom.

Roll Axis (q-Axis): In some embodiments, for wrist pronation-supination, the bicep is used as a grounding to command rotation around the forearm. For each direction of roll, a tendon wrapped around the forearm in a helix extending from the motor and terminating on the wrist mesh is used. An intermediate node is used to maintain the pitch of the helix and a spiral plastic sheath over the tendon allows it to slide smoothly and helps distribute pressure when tensioned.

Based on pilot studies, it was determined that the effectiveness of the roll guidance depends on the pitch of the helix. When the pitch is short, it creates more friction which results in a “squeeze” effect. However, with a longer pitch, there is less friction, making it easier for the tendon to pass through the intermediate nodes and transfer the force to the wrist. Thus, in one embodiment, the pitch is varied as the sleeve expands or contracts with the users' arm lengths, so that the tendon wraps around the arm only once, thus minimizing friction.

Pitch Axis (θ-Axis): In some embodiments, a rack and pinion mechanism along with an SG90 servo motor is used to control the wrist's adduction-abduction. To minimize the weight on the wrist, a smaller servo motor is selected for this particular axis as it was found through pilot studies that a motor with lower torque would be sufficient. The mechanism uses a nylon rod to push or pull on the back of the hand, thus creating the desired motion of the wrist joint. This kinesthetic actuation is made possible by grounding reaction forces on the forearm mesh.

Yaw Axis (ψ-Axis): In some embodiments, kinesthetic guidance is used for wrist ulnar-radial deviation. The motor is placed parallel to the ulnar bone and is grounded on the forearm mesh. It uses a pivot arm to push or pull on the side of the hand. In some embodiments, the pivot arm is held in place by a loosely tensioned Velcro strap, which prevents it from slipping under or over the hand.

Left-Right Translation (X-Axis): In some embodiments, for left-right translation nylon rods were used that connect each side of the forearm mesh to the rack and pinion mechanism attached to the motors on the bicep mesh as shown in FIG. 4. The expansion and contraction of the rods provide the desired guidance. Two nodes per nylon rod are used for support and to prevent buckling during compression.

Up-Down Translation (Z-Axis): In some embodiments, the z-axis haptic guidance operates on the same principle as the x-axis, but the nylon rods run on the top and bottom of the arm.

Forward-Backward Translation (Y-Axis): In some embodiments, all four nylon rods for the X and Z axes are actuated synchronously to achieve a skin-stretch effect on the forearm for longitudinal guidance.

It is important to note that the translation forces delivered along the X and Y axes may appear to act kinesthetically around the elbow joint. However, the experimental results demonstrate that users respond to these cues within the task's reference frame, i.e., the hand, rather than the joint-centric coordinate frame of the elbow. Furthermore, although up-down translation (Z-axis) inherently involves the shoulder joint, the system achieves this motion without explicitly actuating the shoulder. This behavior highlights the system's ability to generate joint torques implicitly by applying skin stretch-based forces to distal links. This novel haptic delivery mechanism is referred to as Referred Torques Mediated by Skin Stretch. A related phenomenon, termed “pseudo-force sensation,” was previously reported where tangential skin deformation evoked a directional force perception in the forearm for two-axis translations (up-down and right-left) (see Y. Kuniyasu, M. Sato, S. Fukushima, and H. Kajimoto, “Transmission of forearm motion by tangential deformation of the skin,” in Proceedings of the 3rd Augmented Human International Conference, ser. AH '12. New York, NY, USA: Association for Computing Machinery, 2012) (see ROS.org contributors, “rosserial: Ros client library for embedded devices,” https://github.com/ros-drivers/rosserial, 2024, accessed: 2025 Apr. 1.)

In some embodiments, the haptic interface is designed to fit users of all arm sizes while allowing free movement. It's constructed on a stretchable nylon sleeve with ratchet mechanisms on each mesh for a snug fit. The tendons are locked in place using screws for easy calibration. The nylon rods can also be adjusted using screws to increase or decrease their length. For the user studies, participant arm measurements ranged as follows: forearm length (25.5-29.5 cm), elbow circumference (22.8-25.4 cm), and wrist circumference (14-16.5 cm).

In some embodiments, the device is manually calibrated. In some embodiments the device calibration can be automated by using actuated nodes that can engage and disengage the tendon and rods. These mechanisms can also be coupled with force feedback sensors to calibrate/adjust the sleeve for different users. By coupling this with force feedback sensors, one can calibrate or adjust the sleeve for individual users. Additionally, using force feedback sensors in combination with closed-loop controls can provide precise control over the forces delivered to the wearer. Feedback mechanisms in addition to force feedback can also be used for calibration. For example, motor current draw can be used to estimate tension in the strings. Further, external position measurement instruments (NDI aurora, NDI Vega, Vicon) could be used. Actuating elements (rods or tendons) can be manually or automatically locked into place on the nodes.

In some embodiments, the rods and/or tethers can be embedded withing the fabric of the sleeve for better rigidity, aesthetics, and ergonomics.

In some embodiments, the device can be manufactured via at least one of sewing, gluing, and/or hot pressing. In some embodiments, the device is manufactured as a single entity. The sleeve may also be eliminated such that the nodes or 3D printed mesh are mounted directly on the user's body. The 3D printed mesh may be replaced with any rigid or semi-rigid component which conforms to the wearer's anatomy at the point of attachment and offers a rigid surface to distribute forces. The surface may itself be flexible but may become rigid after wearing or during actuations to distribute forces.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore specifically point out exemplary embodiments of the present invention and are not to be construed as limiting in any way the remainder of the disclosure.

A pilot study was conducted on three subjects to measure the forces exerted by tendons and nylon rods using a DYMH103 Inline Load Cell (SHANGHAI QIYI Co. Ltd, Shanghai, China). The device weighed 793 grams, excluding the weight of the cable harness, which was suspended from the user's chair to prevent adding any load on the user.

The device was worn by the users during the measurement process to account for any change in output force caused by the effects of friction, buckling of the rods, or the dynamics of the arm's soft tissue. To ensure uniformity in the measurement, the users were instructed to keep their arms still and stretched out with palms facing down. During the calibration of the device, we found that the average pre-tension on the tendons was 3.21 N, while on the rods, it was 2.6 N. The maximum tension applied by the tendons was 17.7 N, and by the rods was 11.6 N. Additionally, the rods were capable of applying a maximum compression of 4.4 N. As discussed herein, a lower torque motor is used for the pitch axis. This axis was compressed to 1.4 N in its neutral position. The maximum compression applied was 4.1 N, while the maximum tension was 1.3 N.

Based on the range of motion of the motors and the sizes of the gears (rack and pinion) and pulleys (for the tendons), the translation rods caused a 33.5 mm displacement, and the tendons caused a 15.7 mm displacement of soft tissue via skin stretch

Seven right-handed users (4 male and 3 female, 20-25 years old) participated in a series of experiments to test the efficacy of ItS-RHAD. The participants wore noise-canceling headphones during the experiments, and the wearable device was covered with a cloth to ensure that only haptic cues were used to guide them.

In the training phase, participants received guidance forces once in each direction, in random order, while being verbally informed of the corresponding direction. In the next round, participants were asked to identify the direction of each cue and were verbally corrected if their responses were incorrect. In an optional third round, only the directions previously misidentified were repeated, with verbal reinforcement provided again. Only 7% of cues in the second round were misinterpreted, requiring a third round, which highlights the intuitiveness of our device. This finding corroborates with our pilot studies where participants intuitively identified the guidance cues without any training.

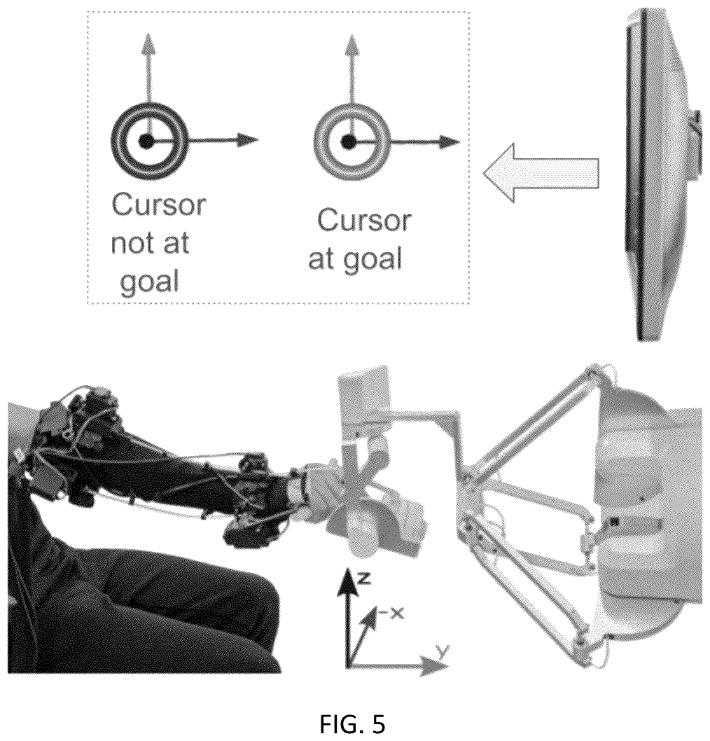

During free-space experiments, Its-RHAD applied maximum force command in each direction. In experiments using the Lambda.7, a proportional control scheme was used such that the commanded force decreases linearly as the user approaches the target. A visual cursor (FIG. 5), comprised a color-changing torus and Cartesian axes, was displayed on a blank screen to indicate hand orientation. It serves only as a visual reference and does not provide guidance cues. The torus starts red and turns green when the user's position or orientation falls within the goal threshold. Users move in the direction of the guidance force until the torus turns green. Force guidance is applied in one direction at a time. Movement is considered complete when the user reaches within 10 mm for translations or 5 degrees for orientations. Guidance forces remain active even after the cursor turns green, and the task is marked complete only if the user remains within the goal threshold for more than 100 ms.

In a correlation experiment, users were asked to identify the direction of haptic cues. This was done to assess the effectiveness of guidance commands given by ItS-RHAD and to test the users' perception of these commands. Initially, this experiment was performed in free space to ensure that the haptic delivery strategy was effective on its own, without any influence from the user's arm being coupled with the haptic device's kinematic chain.

For the remaining experiments, users used the Lambda.7 haptic device (Force Dimension, Nyon, Switzerland) to record their actions. They repeated the correlation experiment and then progressed to perform complex, real-world tasks based solely on haptic guidance from ItS-RHAD. It is noteworthy that users performed these tasks without any contextual knowledge of the exercise, highlighting the efficacy of ItS-RHAD's haptic guidance system.

During free space experiments, the maximum force command in each direction is applied to the user. In experiments where their movements are tracked by Lambda.7, users follow a proportional control scheme where the commanded force decreases linearly as they move closer to the goal point. A cursor, as seen in FIG. 5, comprising a color-changing torus and its cartesian axes, is used as a visual reference. Initially, the torus is red, but turns green when the desired position in the respective axis is within the goal threshold. The users are directed to continue moving in the direction of the guidance force until the cursor turns green. Force guidance is always provided one direction at a time. Therefore, the chosen completion criteria is to move in the commanded direction by a magnitude equal to or greater than the goal position. A threshold of 10 mm for translations and 5 degrees for orientations is used. It is important to note that even when the cursor turns green, guidance force is still applied. The task is considered complete when the user is within the goal threshold for more than 100 ms.

### Correlation Task:

During the free space experiment, participants were given random guidance forces, while their verbal responses were recorded in two sets. Later, they repeated the experiment using the Lambda.7 haptic device. The Lambda.7 provided haptic feedback based on a spring-damper scheme to guide participants back to the starting pose after achieving the goal in each direction or if the response time exceeded the 10-second limit.

In the free space part of this experiment, a movement is considered complete upon receiving verbal confirmation from the user. In the part where the Lambda.7 haptic device tracks movements, users are guided to move 40 mm in each translational direction and 20 degrees in each orientation. A movement is complete when the user reaches or exceeds the target displacement in the specified direction.

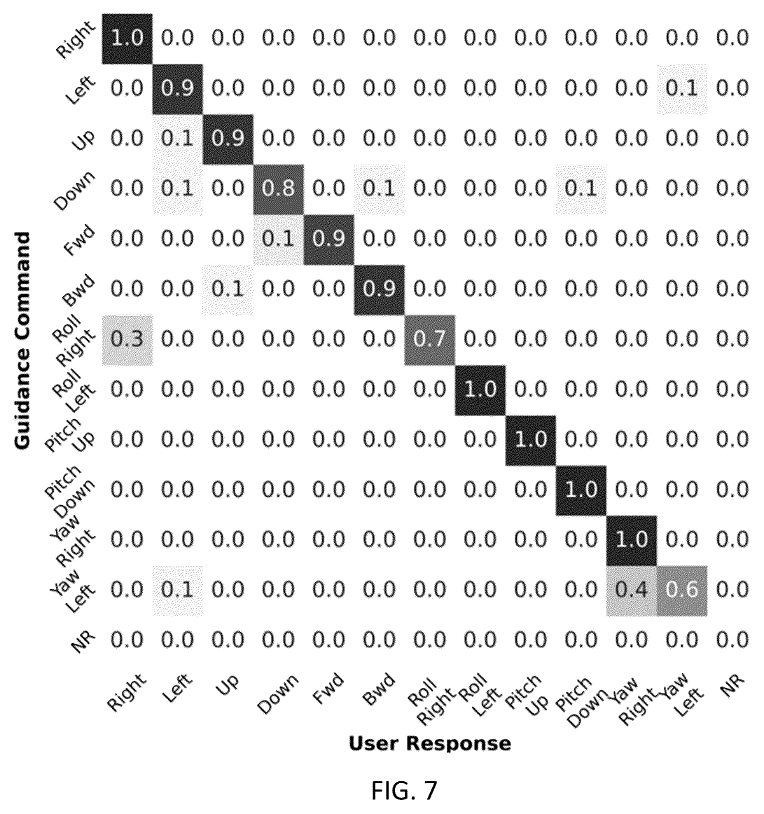

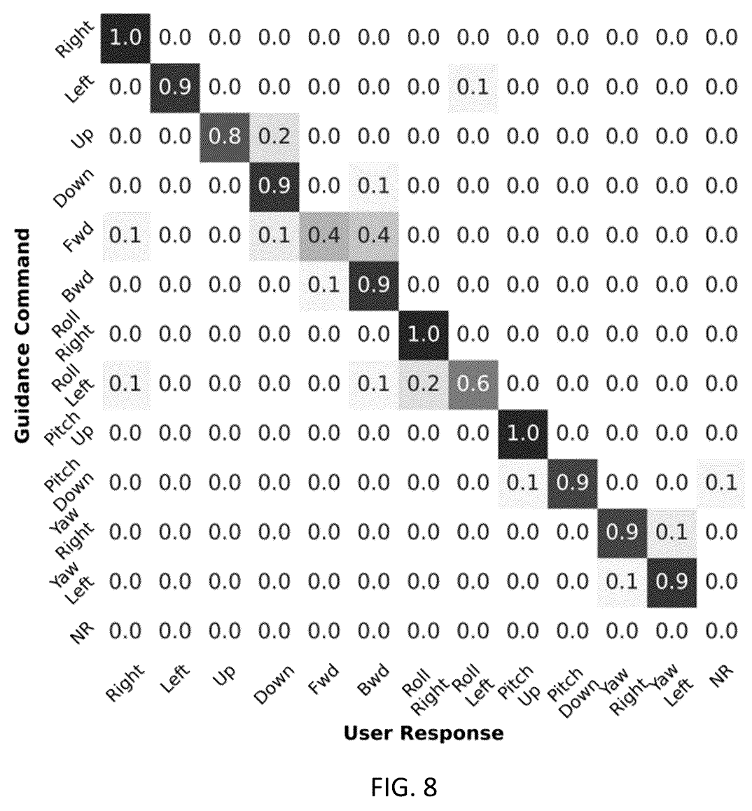

The confusion matrix in FIG. 7 shows that for 168 guidance commands given in each direction, users identified the directions with 89.3% accuracy when operating in free space while FIG. 8 shows that the accuracy dropped to 85.5% when their motion was tracked using the Lambda.7 haptic device.

Both conditions demonstrated high accuracy, with McNemar's test (p=0.324) and the Wilcoxon Signed-Rank test (p=0.250) indicating no statistically significant difference between them. This confirms that coupling with the Lambda.7 did not significantly impact haptic feedback delivery, supporting the consistency of the haptic system across both conditions.

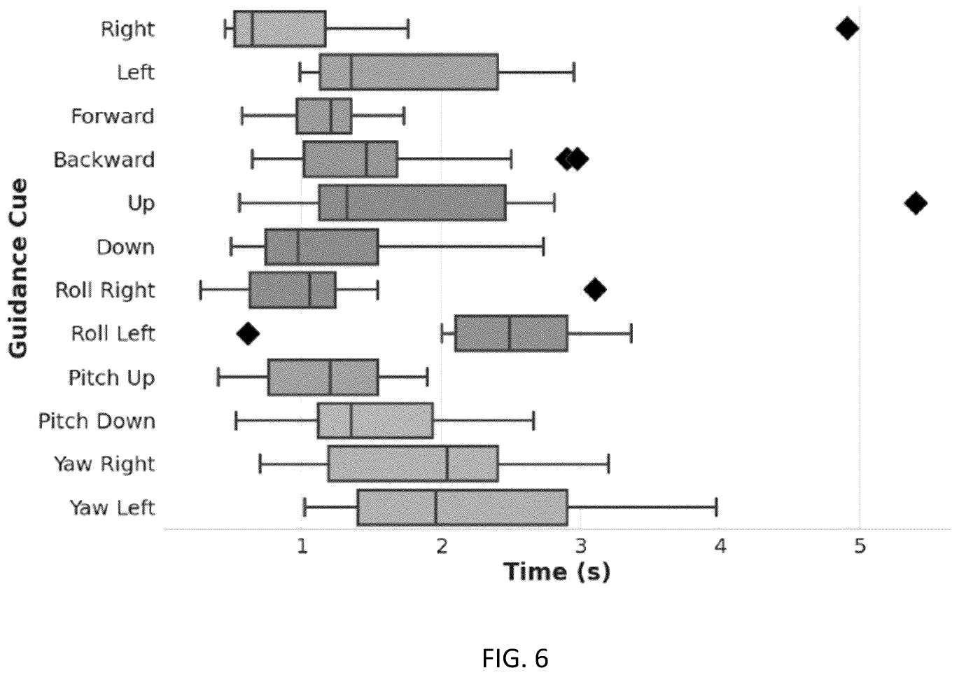

Response time was defined as the time between initiating the haptic guidance and when the participant began moving in the correct direction. Only the correctly identified responses in the experiment with Lambda.7 were used to calculate the response time, as the free space correlation feedback was verbal. The box plot in FIG. 6 displays the average response time for each axis, which was 1.36 seconds. The fastest response time was 0.27 seconds, while the slowest was 5.4 seconds. A one-way ANOVA showed a statistically significant difference in response times only for the Roll Right vs. Roll Left pair (p=0.0004). All other pairs (e.g., Left-Right, Up-Down, etc.) showed no significant differences (p≥0.094).

### Drawing Task:

The task was divided into four continuous components, each providing guidance in one translation direction. Following these sub-tasks sequentially, users were able to draw a square with sides of 80 mm in a counter-clockwise direction. As the guidance force was unidirectional, success was achieved if users were able to move from their position at the end of the previous sub-task to any point on the desired edge, even if their trajectories did not trace a perfect square.

Users were not given any prior information about the task. They were asked to draw in both XY and XZ planes, with the plane in which they drew first being alternated between subjects. A red torus was provided, which turned green after reaching each vertex. When the torus turned green, users were instructed to hold their hand pose for two seconds. After that, the guidance force changed directions to “draw the next edge,” and the color of the torus changed back to red.

To record users' movements, the Lamba.7 was used, and haptic feedback was provided to constrain users in the desired plane and track their movement. The task had a time limit of one minute, and if users failed to achieve all vertices within this time, the task was considered incomplete.

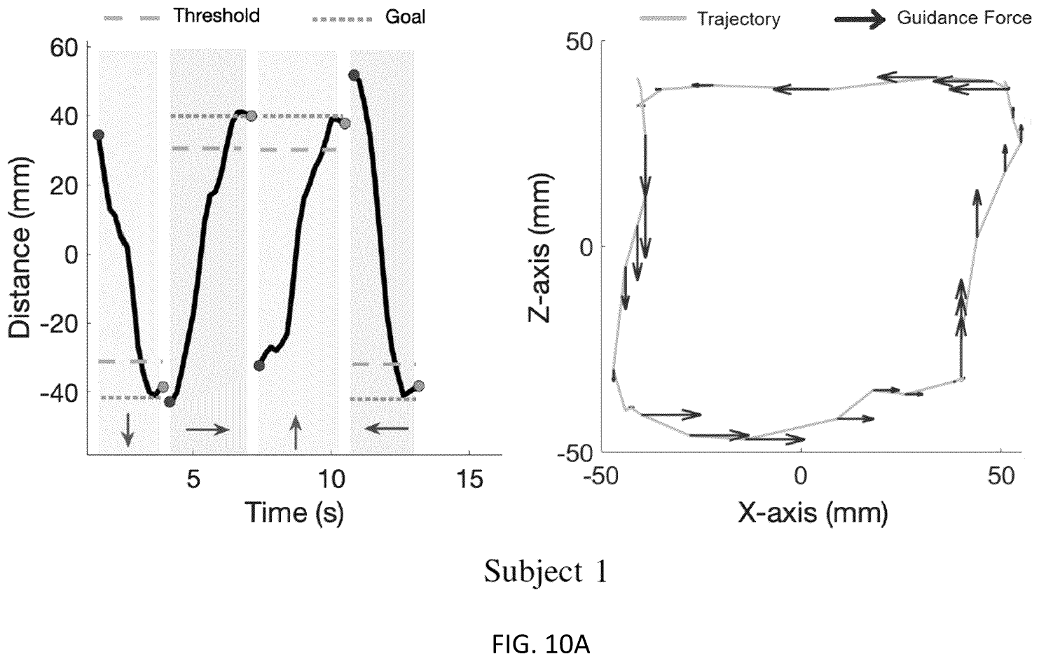

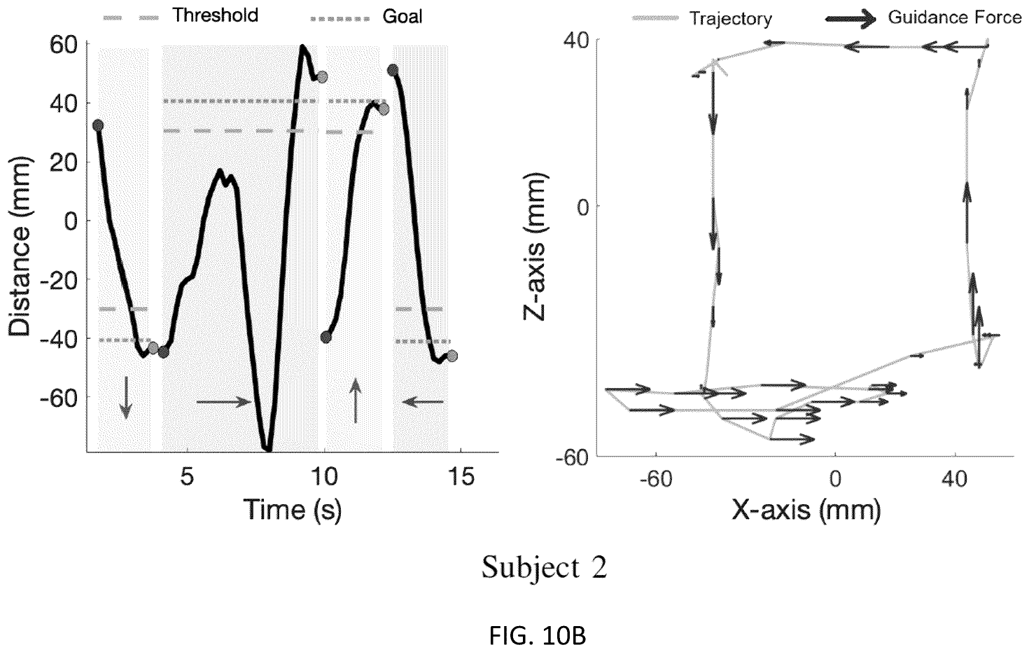

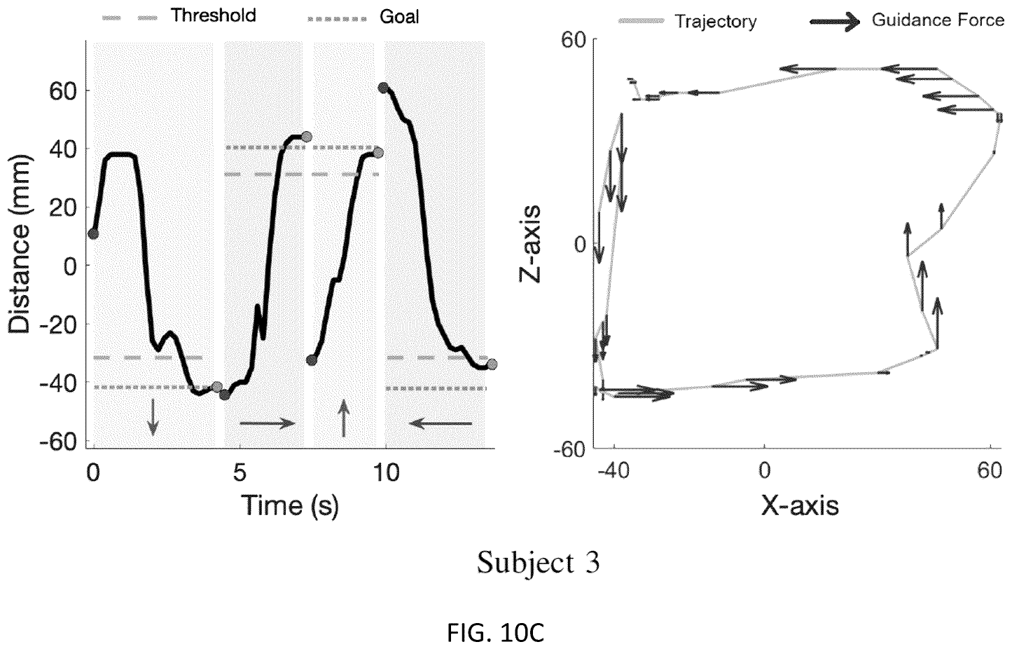

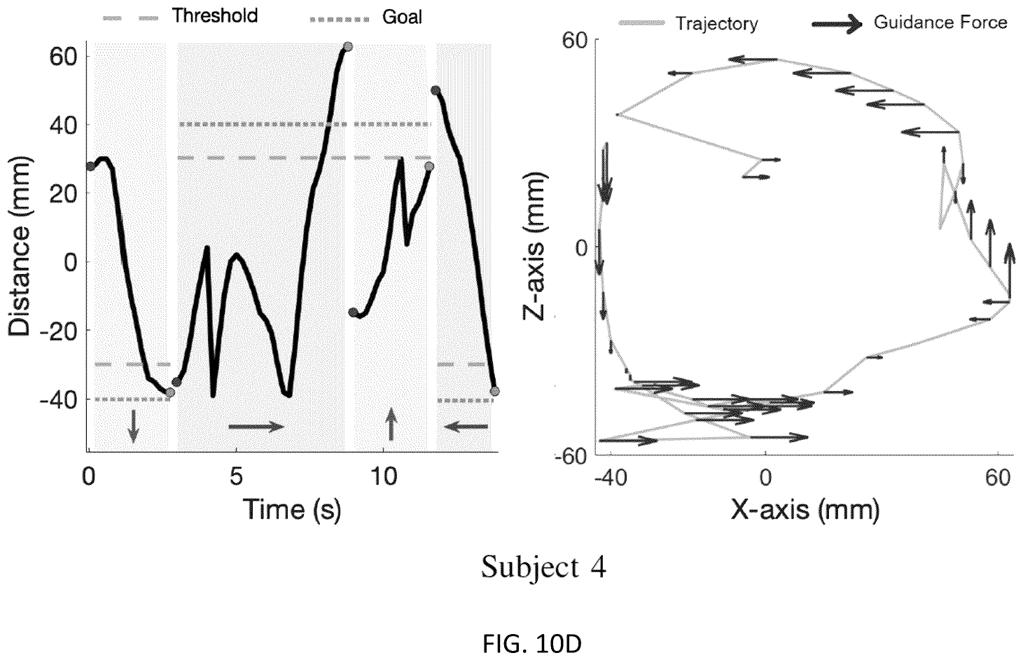

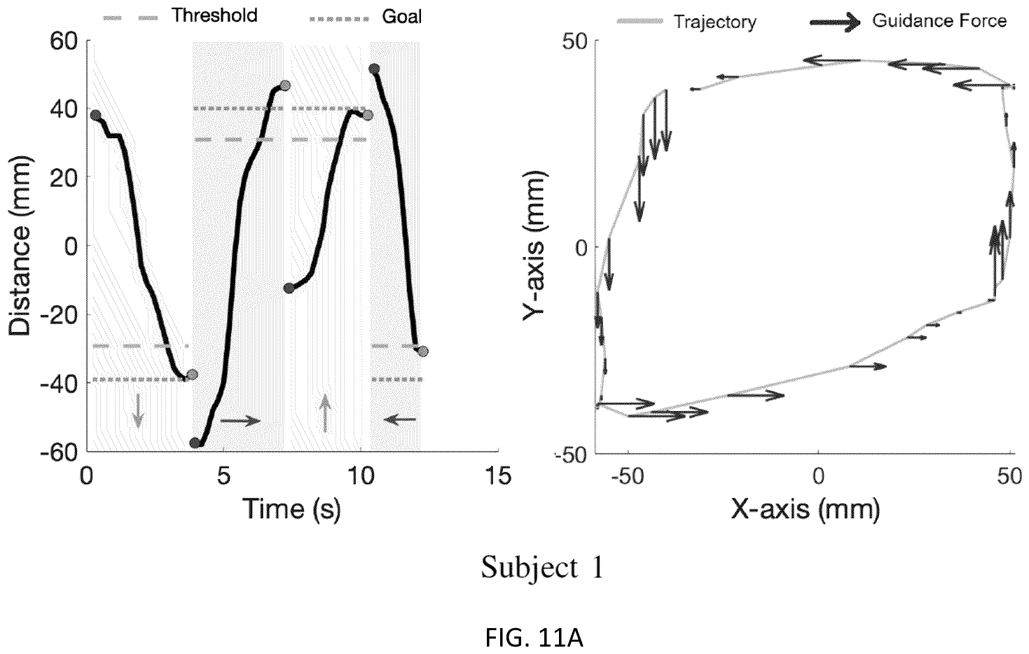

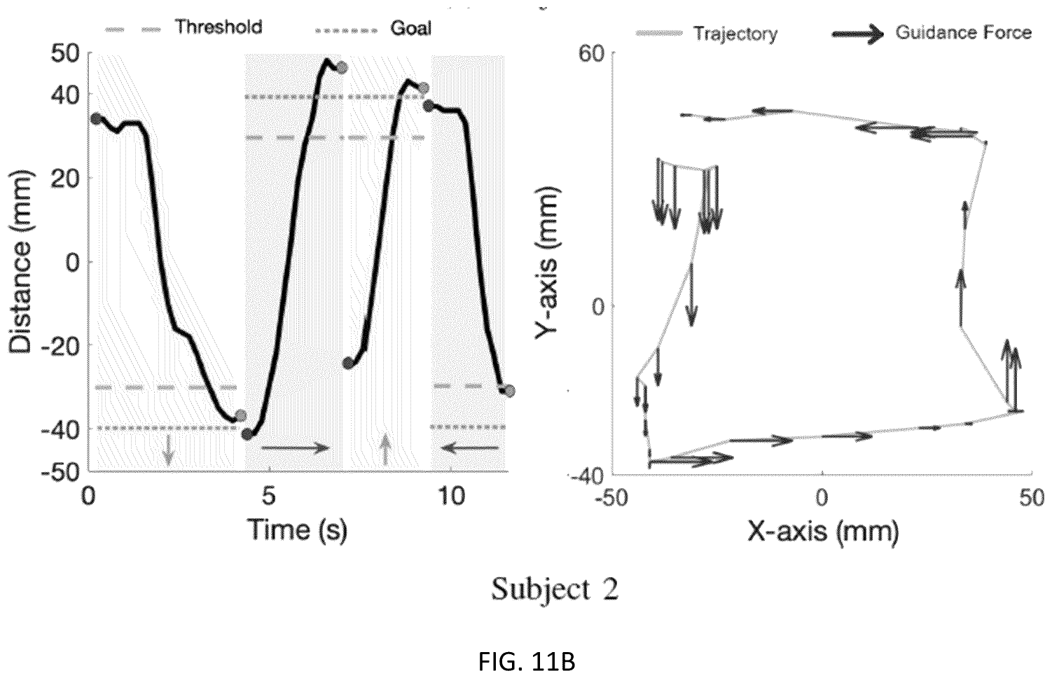

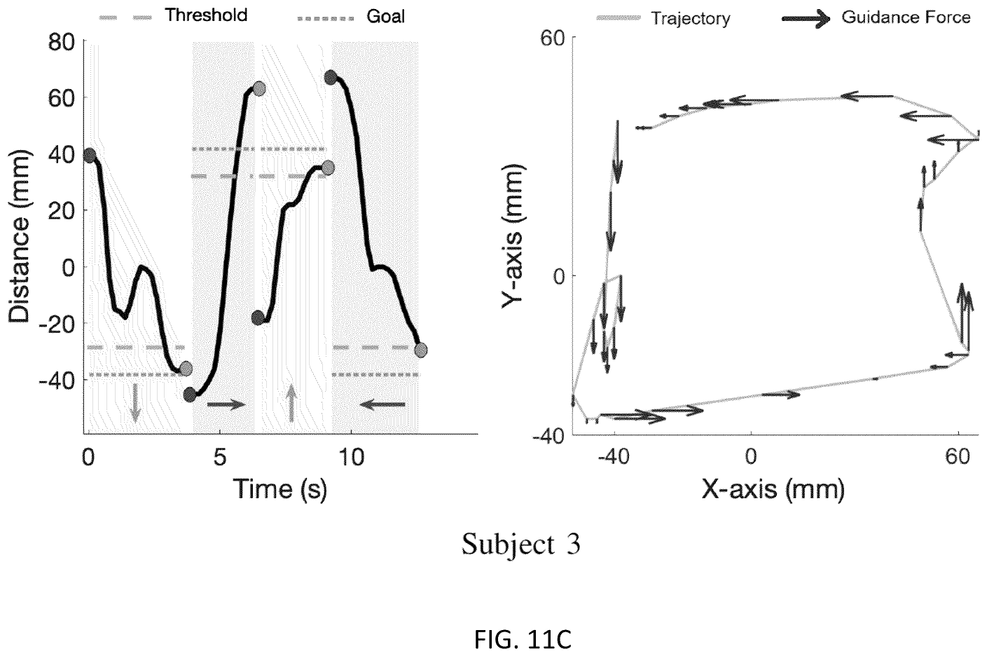

The task was difficult to complete as it required all vertices to be reached within a given amount of time. Four subjects successfully drew squares in the XZ plane, while three succeeded in the XY plane. The results from each subject are represented in FIGS. 10A-10D and FIGS. 11A-11C, respectively. Their trajectories in the respective plane are shown in the right column, with pink arrows indicating the direction and magnitude of the guidance force provided by ItS-RHAD in each state.

The left column displays the trajectory of each subject along each edge of the square. The trajectory is represented by red, green, and blue colors for the x, y, and z axes in the task space, respectively. Ideally, the trajectories should be smooth and monotonic slopes based on the direction of motion. However, some of the actual trajectories show a non-monotonic nature, indicating that these users started in the right direction but lacked confidence in their chosen direction, causing them to reverse directions. Every time a user reversed direction and moved a distance greater than the completion threshold in the opposite direction, that was counted as a direction reversal. This behavior was most frequently observed during the “right” command for subject 2, as shown in FIG. 10B. The subject initially started moving in the correct direction but lost confidence in their decision, causing them to change direction. They then reversed direction again and finally reached the goal point. Overall, in the group that successfully completed the drawing tasks, subjects reversed their direction 13 times out of 28 total guidance commands. Most of these reversals occurred during the “right” command of the XZ square. Excluding these commands, there were only 5 reversals out of 24 commands. There were no reversals in the left and forward commands.

The average error for each edge in the XZ plane was 6 mm, with a Standard Deviation (S.D.) of 3.35 mm. On the other hand, in the squares of the XY plane, the average error was 5.58 mm, with a S.D. of 2.91 mm. However, for both planes, the errors for the “right” guidance command were much higher compared to the other axes, with an average error of 12 mm and a S.D. of 9.21 mm. If we exclude the errors for the “right” command, the average position error for both planes combined was 3.81 mm, with a S.D. of 1.98 mm.

Ideally, trajectories should be smooth and monotonic, following the intended direction of motion. However, some participants exhibited non-monotonic trajectories, reversing direction after starting correctly. A reversal was counted whenever a participant moved more than the completion threshold in the opposite direction. Across all participants who successfully completed the task, there were 13 direction reversals out of 28 guidance commands. Excluding the “right” command, there were only 5 reversals out of 24 commands. No reversals occurred for the “left” or “forward” commands.

The Mann-Whitney U test (p=0.853) showed that task order (XY-first or XZ-first) did not significantly affect performance. Overall, these results demonstrate that the haptic delivery methods effectively guided users to desired positions and enabled them to complete complex tasks requiring precise sequential movements.

Out of fourteen tasks, only seven were completed successfully. It is believed that this was due to the high level of difficulty of the task, as even missing one vertex resulted in an incomplete task. Additionally, the guidance command's direction changed too abruptly from one phase to the next, making it difficult for participants to understand and follow. Out of all the attempts that failed to complete the challenge, two failures occurred because the participants were unable to reach the second vertex, two because they could not reach each of the second and third vertices, and one because the participant moved in the opposite direction of the command and was unable to reach the final vertex.

The successful attempts from this experiment proved that ItS-RHAD effectively guided wearers with an average position error of 5.79 mm and an S.D. of 7.21 mm. This indicates that the interface can accurately guide wearers to perform a series of translation movements with sub-centimeter-level precision.

Based on the results of this task, it is clear that the disclosed haptic delivery method can guide users to a desired position in any direction. Furthermore, the disclosed delivery method has proven to be effective in guiding users to complete a series of movements required to accomplish a meaningful task.

### Compound Task:

In this experiment, the haptic delivery system's ability to guide users through tasks requiring both orientation and translation motion was tested. To achieve this, three tasks were created inspired by everyday movements and modeled by a simple buzz-wire game [see P.Q. Lee et al., 2022]. The tasks included opening and closing a door, screwing and unscrewing a pickle jar, and making a paintbrush stroke. Each task required two orientations and two translations.

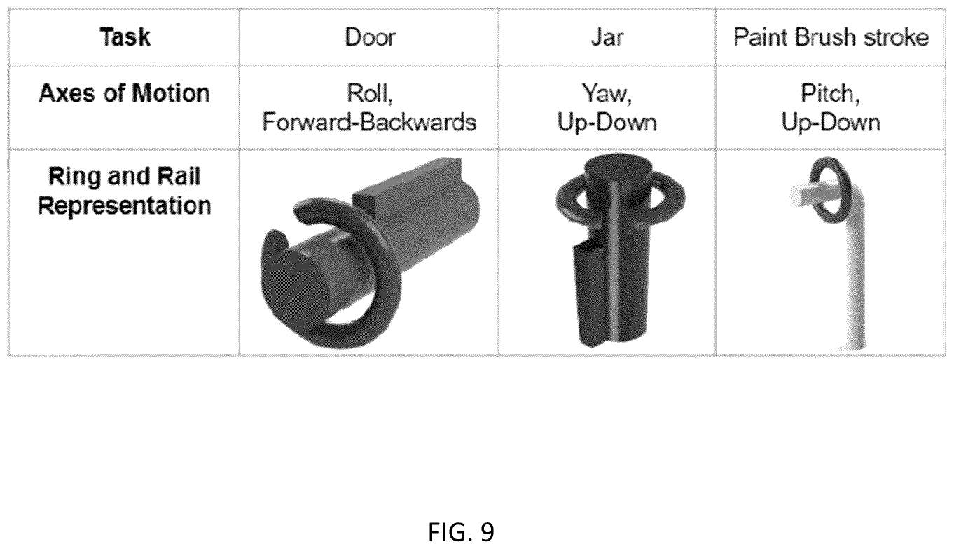

In the virtual buzz-wire game (shown in FIG. 9), the red notched ring represents the user's hand, and the cylinder represents the wire. The Lambda.7 provided environmental haptic constraints to ensure the loop remained centered around the wire. Additionally, the interaction of the notch in the loop and the key or bend in the cylinders added orientation constraints to the loop.

Users were solely guided through haptic guidance provided by ItS-RHAD and had no knowledge about the task being performed. The tasks are designed as follows:

Door Task: In this task, users unlock a doorknob (roll right), push open the door (forward), pull the door shut (backward), and lock the doorknob (roll left). As shown in FIG. 9, there is a virtual key on the cylinder that limits the forward-backward movement until the slot on the ring aligns with the key. This simulates the interaction with the door frame and allows the user to unlock the door. After that, during the push/pull phases, the orientation in the roll axis is constrained, while the ring can slide forward-backward over the cylinder. This simulates the user holding the doorknob in the unlocked position while pushing and pulling on the door. Lastly, during the final roll left movement, forward-backward translation is constrained again, simulating the interaction with the door frame while locking the doorknob.

Jar Task: The jar task involves unscrewing the lid (yaw left), lifting it up (translate up), replacing the lid (translate down), and screwing it back on (yaw right). Similar to the door task, there is a virtual key on the cylinder that limits the up-down movement until the slot on the ring lines up with the key. This signifies that the lid cannot be lifted unless it is unscrewed. After unscrewing the lid, orientation in the yaw axis is restricted during the lifting and replacing phases, while the ring can slide up and down over the cylinder. This simulates the user holding the lid in the unscrewed position while lifting it up and replacing it on the jar. Lastly, during the final yaw right movement, up-down translation is constrained again, simulating the interaction of the lid with the jar.

Paintbrush Task: In this task, users are required to point their hand down (pitch down), paint a descender stroke (translate down), paint an ascender stroke (translate up), and return the hand to the nominal orientation (pitch up). The buzz-wire representation in FIG. 9 illustrates the task. In the first phase, the motion along the up-down axis is restricted. However, once the pitch down motion is completed, this constraint is unlocked. During the ascender/descender strokes, the ring is allowed to slide up and down the cylinder while the pitch is constrained. Finally, in the last stage, the up-down translation is constrained again. Imaginary constraints are used in this task based on how a painter might orient their hand during each stage of performing a brush stroke.

### Procedure:

In this study, participants were asked to complete a set of compound tasks consisting of four sub-tasks. Each sub-task provided haptic guidance commands in a specific direction, and the users' motion was tracked using Lambda.7. The cursor turned green after each stage for 200 milliseconds and turned back to red when the next direction command was given. Haptic constraints were provided as described in each of the tasks. The participants were given one minute to complete each task, and if they failed to complete all phases within that time frame, the task was considered incomplete. The order in which the participants performed the tasks was randomized.

### Statistical Analysis Methods:

For the correlation experiments, normalized confusion matrices were plotted for both conditions: in free space and when coupled with the Lambda.7 haptic device. Confidence intervals were then calculated for each confusion matrix. Statistical analysis was conducted to evaluate the accuracy difference between the Lambda and Free Space conditions to ensure that coupling with the grounded haptic device did not influence the device's haptic delivery. McNemar's test and the Wilcoxon Signed-Rank test were used to compare the two datasets, assessing difference in accuracy between the conditions. One-way ANOVA was used to compare the mean response times for each directional pair (e.g., Left-Right, Up-Down) to determine whether users' response times varied significantly across specific cues.

For the planar Drawing task, the Mann-Whitney U test was used to evaluate whether the sequence of experiments (XY-first vs. XZ-first) affects user performance.

In the three compound tasks, the Wilcoxon Signed-Rank Test was used to compare the errors and the number of direction reversals for each directional pair. Additionally, Spearman Correlation was used to study the relationship between the magnitude of errors and the number of direction reversals for each motion axis.

A significance threshold of p<0.05 was used for all tests.

### Results:

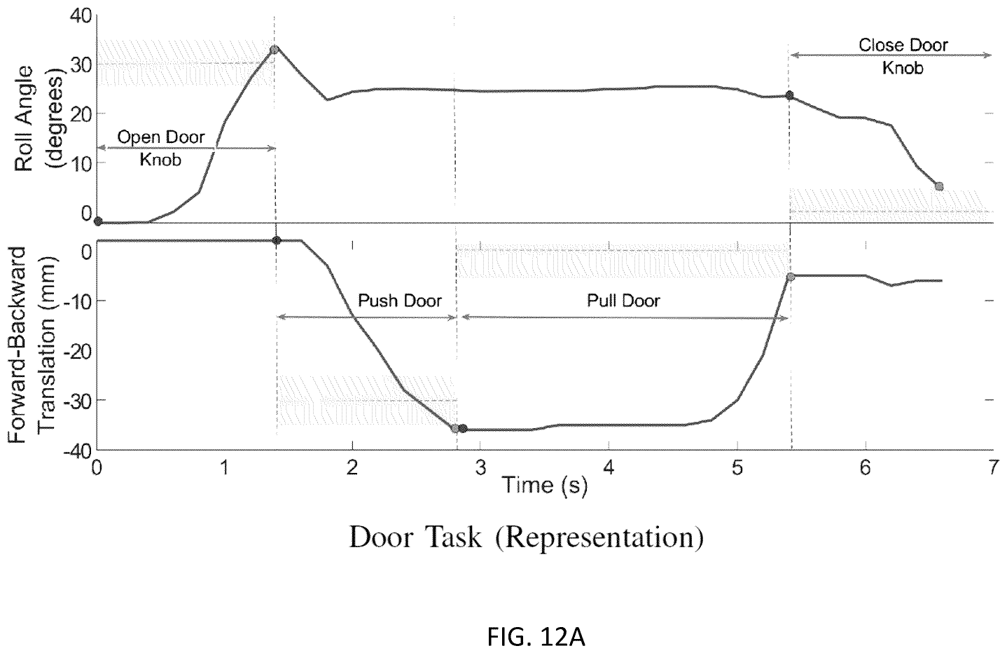

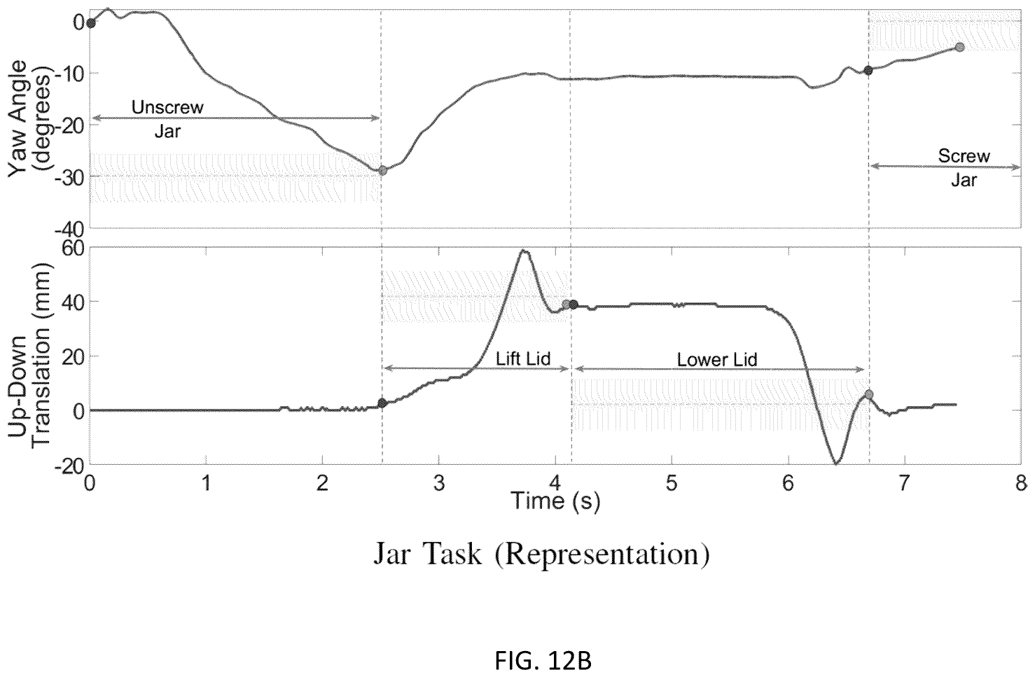

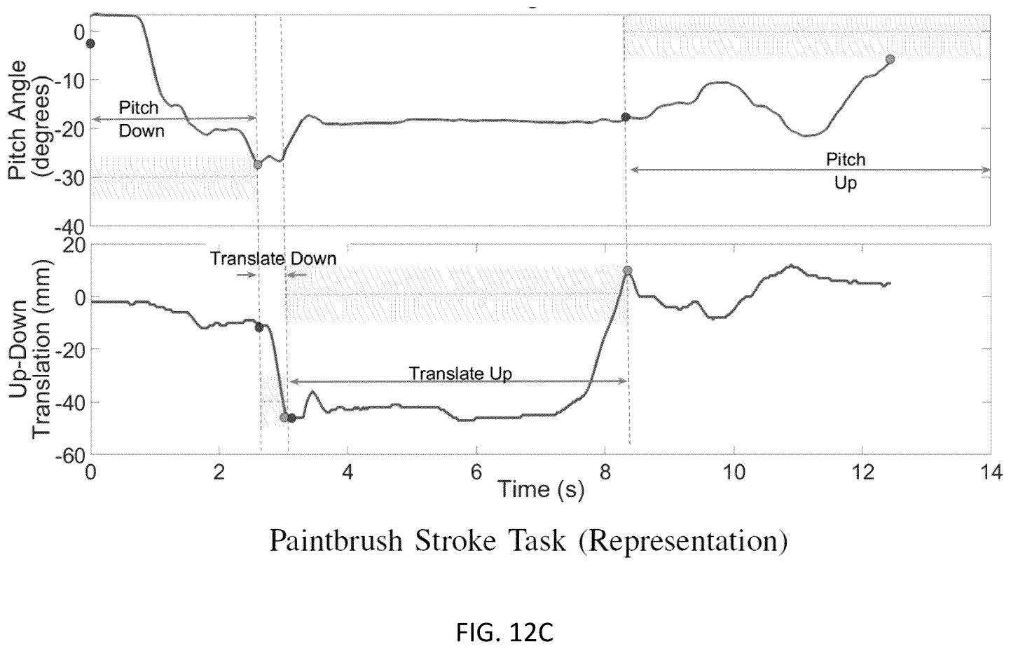

FIGS. 12A-12C show the path of the best-performing participants in each task based on the least number of reversals. All participants successfully completed the compound tasks.

In the door task, the average position error for the roll-right axis was 4.67 degrees, with a S.D. of 1.76 degrees. For the forward-backward translation, the mean position error at the goal was 2.43 mm with an S.D. of 0.99 mm.

The average yaw position error in the jar task was 3.34 degrees with an S.D. of 1.247 degrees, while for the paintbrush task, the average pitch position error was 3.9 degrees with an S.D. of 1.4 degrees.

Combining the results for the jar and paintbrush task, the average error for up-down translation was 4.01 mm with an S.D. of 9.19 mm.

When a user reversed direction and moved a distance greater than the completion threshold in the opposite direction, it was counted as a direction reversal. For rolling right, the average number of direction changes was two, with a maximum of four for one subject. When moving forward, subjects reversed directions an average of one time, while when moving backward, only one subject reversed direction three times. None of the subjects reversed directions while rolling left.

In the jar task, the average number of reversals while yawing left was two, with an S.D. of one. Only one subject reversed direction once while yawing right. For the up translation, the average number of direction changes was one, while for the down translation, two subjects reversed direction once each.

In the pitch motion of the paintbrush task, the average reversal for pitch down was one with an S.D. of one. Additionally, two subjects reversed direction once each for both downward translation and pitch up. One subject reversed direction three times, and another reversed once in the upward translation.

The Wilcoxon Signed-Rank Test showed statistically significant differences between the errors for the Roll Right vs. Roll Left (p=0.0260) and the Yaw Right vs. Yaw Left (p=0.0422) pairs. For the number of direction reversals, differences were found for the Right vs. Left (p=0.0277) and the Pitch Up vs. Pitch Down (p=0.0156) pairs. The Spearman correlation showed no significant relationship between the magnitude of errors and the number of direction reversals for any axis.

In overview, three experiments on haptic navigation were performed involving seven participants. The users were able to accurately identify haptic guidance commands for hand movements such as roll, pitch, yaw, and left-right, up-down, forward-backward translations within a reasonable response time. This demonstrates the initial feasibility of the wearable haptic interface in providing proprioceptive haptic guidance.

Slightly higher accuracy was observed when users interacted independently with the wearable haptic interface in free space as compared to when they used a closed-chain grounded haptic device. However, statistical analysis revealed no significant difference between the free space and Lambda.7 correlation tasks. The pilot studies using both the Lambda.7 and the Geomagic Touch (3D Systems, South Carolina, USA), were consistent with this observation. It is believed this difference in accuracy is due to the limitations created by the workspace of the haptic device on the user's arm, which may restrict free movement in certain arm positions. The fact that the haptic interface guides the wearer in the task space (their hand) instead of the joint space of the arm may also contribute to this behavior, as there is no way to check if a commanded pose is achievable. However, this is not considered as a limitation of the haptic interface since it is not expected for wearers to interact with other kinematic chains. Grounded haptic devices were only used for experimental evaluation purposes.

In the correlation test, participants could identify the axis they were supposed to move in correctly. However, they sometimes moved in the opposite direction of the command. This could be due to the proportional control scheme, which caused the tension to decrease as the user moved in the desired direction. Consequently, the guidance command might have felt like it was in the opposite direction. In hindsight, a velocity-based control scheme [see B. Mathur et al., 2019] may have yielded better results.

In the latest experiment, real-world tasks were utilized to help users perform complex movements. With the use of the wearable haptic interface, participants were guided through these complex tasks with an orientation accuracy of 3.97 degrees with an S.D. of 2.2 degrees, and a position accuracy of 3.22 mm, with an S.D. of 7.32 mm. This confirmed that the wearable haptic interface has the capability to guide users with high precision in executing a series of complex tasks, which involve both translations and orientations.

In a post-experiment survey, participants were asked to guess which tasks they had performed. Interestingly, six participants correctly associated their movements with the task of opening and closing a door, while four accurately identified the jar task. Although three participants could recall their movements in the paintbrush task, they could not relate them to any practical task in the real world.

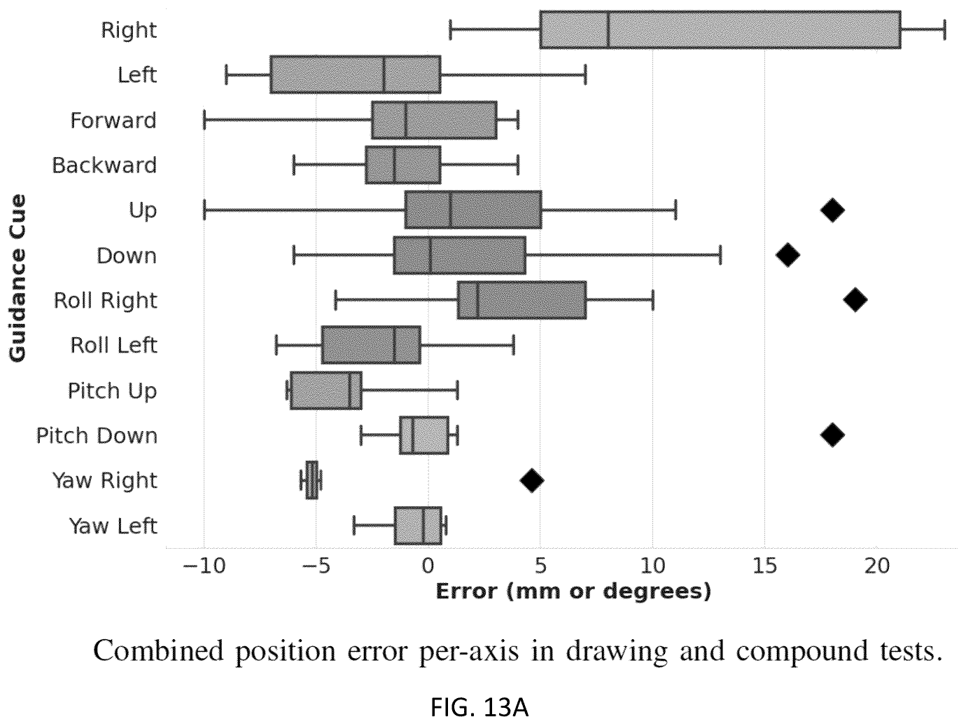

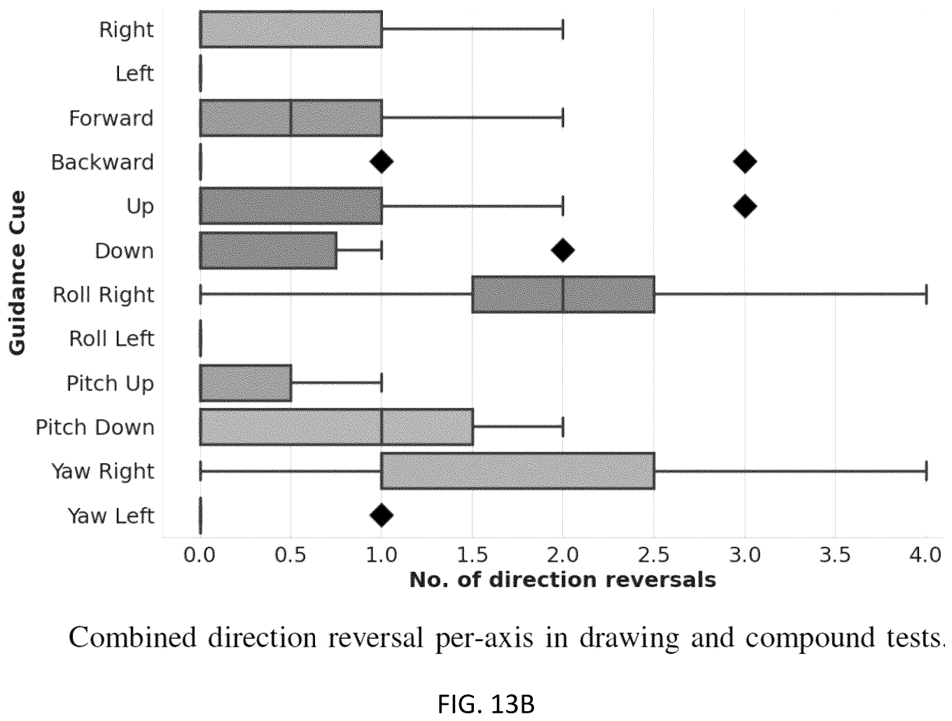

FIGS. 13A-13B display the results of the combined analysis regarding the movements made in successful attempts of the drawing and compound tasks. The analysis indicates that the greatest position error of 23.4 mm was observed during the right translation. This error only occurred during the drawing task, as left-right translations were not tested during the compound tasks. It arose from the seven times the right direction was commanded during the drawing task.

During the experiments, it was observed that the sleeve on the wearer's arm moved, causing the maximum number of reversals in the yaw and roll right directions. According to the correlation tests, this shift caused the perception of a roll right as a translate right, and the yaw right was too weak to be sensed. To fix these issues, improvements to the fixation of the forearm mesh easily solves the problems.

For the remaining axes, participants reversed direction less than once on average. At least one participant had fewer reversals in an axis in the direction that they encountered later. For example, if the subject had already performed the right movement for that task, the probability of reversal going left would be lower. This is ostensibly because it is easier to discern the commanded force once its opposite counterpart has already been encountered.

In conclusion, disclosed herein are details of an exemplary design, fabrication methods, and evaluation of a low-cost (less than $100) novel ungrounded wearable haptic interface that can provide proprioceptive guidance in six degrees of freedom. The interface utilizes kinesthetic feedback to accurately guide the wrist joint movements, while also employing skin stretch to precisely control wrist pronation-supination. Furthermore, disclosed is a novel haptic delivery mechanism, known as “Referred Torques Mediated by Skin Stretch”, and its use to effectively guide translation movements. An ungrounded haptic force delivery mechanism was created by grounding the actuators effectively to evenly distribute reaction forces. This grounding surface also provided a rigid surface to mount the actuators, which were positioned on a part of the arm different from the one being guided.

A device called ItS-RHAD was developed to evaluate the effectiveness of the haptic delivery methods described above in providing precise ungrounded haptic guidance. Two types of user studies were conducted to investigate the performance of ItS-RHAD. In the first study, users identified the direction in which the commanded force was rotating or translating their hand. In the next set of studies, users performed several real-world tasks consisting of a series of sequential movements combining translations and orientations. The wearable haptic delivery methods can provide highly precise guidance, accurate to sub-centimeter levels in translations and within a precision of approximately five degrees in orientation. These findings demonstrate the early feasibility of the technology, which can be significantly improved through more sophisticated control techniques and further refinements in its fabrication.

A wearable system like this could potentially guide surgeons in using navigated surgical instruments such as probes, drills, and saws. This would provide a low-cost and low-footprint option as compared to grounded robotic surgical systems. It could also be used for surgical training and in other Virtual Reality applications requiring dynamic haptic guidance and feedback.

### Computing Environment

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

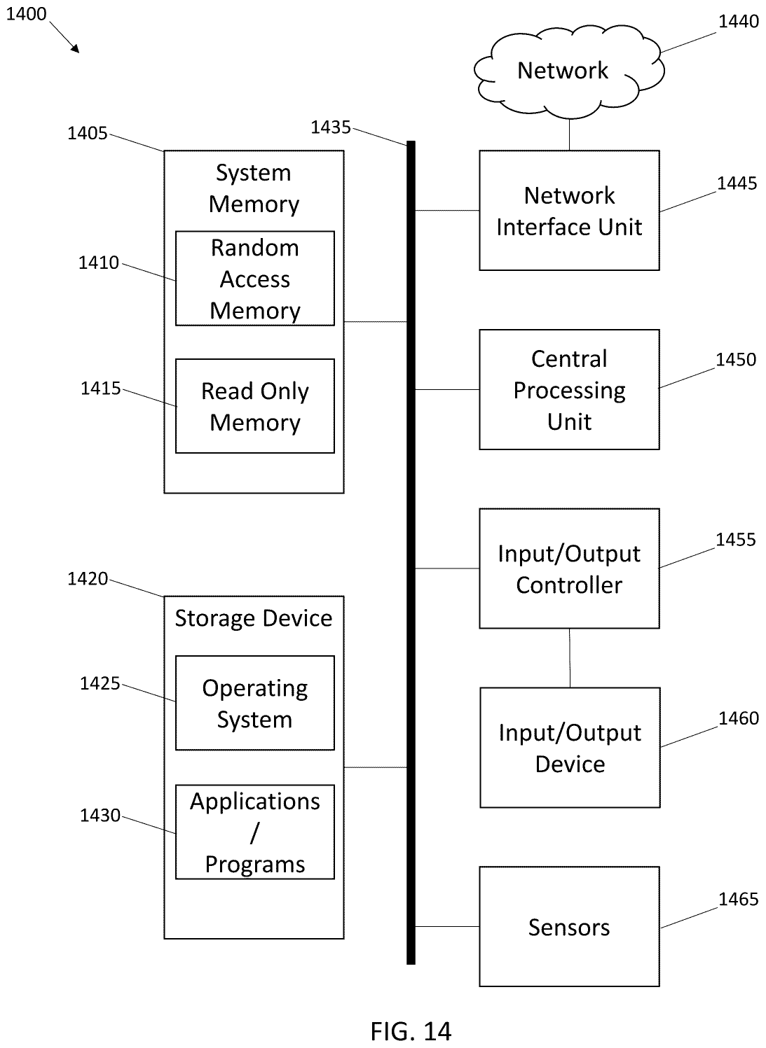

FIG. 14 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 14 depicts an illustrative computer architecture for a computer 1400 for practicing the various embodiments of the invention. The computer architecture shown in FIG. 14 illustrates a conventional personal computer, including a central processing unit 1450 (“CPU”), a system memory 1405, including a random-access memory 1410 (“RAM”) and a read-only memory (“ROM”) 1415, and a system bus 1435 that couples the system memory 1405 to the CPU 1450. A basic input/output system containing the basic routines that help to transfer information between elements within the computer, such as during startup, is stored in the ROM 1415. The computer 1400 further includes a storage device 1420 for storing an operating system 1425, application/program 1430, and data.

The storage device 1420 is connected to the CPU 1450 through a storage controller (not shown) connected to the bus 1435. The storage device 1420 and its associated computer-readable media, provide non-volatile storage for the computer 1400. Although the description of computer-readable media contained herein refers to a storage device, such as a hard disk or CD-ROM drive, it should be appreciated by those skilled in the art that computer-readable media can be any available media that can be accessed by the computer 1400.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 1400 may operate in a networked environment using logical connections to remote computers through a network 1440, such as TCP/IP network such as the Internet or an intranet. The computer 1400 may connect to the network 1440 through a network interface unit 1445 connected to the bus 1435. It should be appreciated that the network interface unit 1445 may also be utilized to connect to other types of networks and remote computer systems.

The computer 1400 may also include an input/output controller 1455 for receiving and processing input from a number of input/output devices 1460, including a keyboard, a mouse, a touchscreen, a camera, a microphone, a controller, a joystick, or other type of input device. Similarly, the input/output controller 1455 may provide output to a display screen, a printer, a speaker, or other type of output device. The computer 1400 can connect to the input/output device 1460 via a wired connection including, but not limited to, fiber optic, ethernet, or copper wire or wireless means including, but not limited to, Bluetooth, Near-Field Communication (NFC), infrared, or other suitable wired or wireless connections.

As mentioned briefly above, a number of program modules and data files may be stored in the storage device 1420 and RAM 1410 of the computer 1400, including an operating system 1425 suitable for controlling the operation of a networked computer. The storage device 1420 and RAM 1410 may also store one or more applications/programs 1430. In particular, the storage device 1420 and RAM 1410 may store an application/program 1430 for providing a variety of functionalities to a user. For instance, the application/program 1430 may comprise many types of programs such as a word processing application, a spreadsheet application, a desktop publishing application, a database application, a gaming application, internet browsing application, electronic mail application, messaging application, and the like. According to an embodiment of the present invention, the application/program 1430 comprises a multiple functionality software application for providing word processing functionality, slide presentation functionality, spreadsheet functionality, database functionality and the like.

The computer 1400 in some embodiments can include a variety of sensors 1465 for monitoring the environment surrounding and the environment internal to the computer 1400. These sensors 1465 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

### REFERENCES

The following publications are each hereby incorporated herein by reference in their entirety:
C. D'Ettorre, A. Mariani, A. Stilli, F. Rodriguez y Baena, P. Valdastri, A. Deguet, P. Kazanzides, R. H. Taylor, G. S. Fischer, S. P. DiMaio, A. Menciassi, and D. Stoyanov, “Accelerating Surgical Robotics Research: A Review of 10 Years With the da Vinci Research Kit,” IEEE Robotics & Automation Magazine, vol. 28, no. 4, pp. 56-78, December 2021.
A. Attanasio, B. Scaglioni, E. De Momi, P. Fiorini, and P. Valdastri, “Autonomy in surgical robotics,” Annual Review of Control, Robotics, and Autonomous Systems, vol. 4, no. 1, pp. 651-679, 2021.
P. Culmer, A. Alazmani, F. Mushtaq, W. Cross, and D. Jayne, “Haptics in surgical robots,” in Handbook of Robotic and Image-Guided Surgery, M. H. Abedin-Nasab, Ed. Elsevier, January 2020, pp. 239-263.
J. Cobb, J. Henckel, P. Gomes, S. Harris, M. Jakopec, F. Rodriguez, A. Barrett, and B. Davies, “Hands-on robotic unicompartmental knee replacement,” The Journal of Bone & Joint Surgery British Volume, vol. 88-B, no. 2, pp. 188-197, 2006.
C. J. Payne and G.-Z. Yang, “Hand-held medical robots,” Annals of Biomedical Engineering, vol. 42, no. 8, pp. 1594-1605, 2014.
G. J. Kane et al., “System design of a hand-held mobile robot for craniotomy,” Medical Image Computing and Computer-Assisted Intervention (MICCAI), vol. 12, pp. 402-409, 2009.
S.-S. Hung, A. S.-F. Hsu, T.-H. Ho, C.-H. Chi, and P.-L. Yen, “A robotized handheld smart tool for orthopedic surgery,” The International Journal of Medical Robotics and Computer Assisted Surgery, vol. 17, no. 5, p. e2289, 2021.
F. Focacci, M. Piccigallo, O. Tonet, G. Megali, A. Pietrabissa, and P. Dario, “Lightweight hand-held robot for laparoscopic surgery,” in Proceedings 2007 IEEE International Conference on Robotics and Automation, April 2007, pp. 599-604.
J. Shang et al., “An articulated universal joint based flexible access robot for minimally invasive surgery,” in 2011 IEEE International Conference on Robotics and Automation, May 2011, pp. 1147-1152.
R. J. Hendrick, S. D. Herrell, and R. J. Webster, “A multi-arm handheld robotic system for transurethral laser prostate surgery,” in 2014 IEEE International Conference on Robotics and Automation (ICRA), May 2014, pp. 2850-2855.
X. Luo, J.-T. Lin, and T. K. Morimoto, “HaPPArray: Haptic Pneumatic Pouch Array for Feedback in handheld Robots,” in 2023 IEEE International Conference on Robotics and Automation (ICRA), May 2023, pp. 12437-12442.
L. R. Reyes, P. Gavino, Y. Zheng, J. Boehm, M. Yeatman, S. Hegde, C. Park, E. Battaglia, and A. M. Fey, “Towards telementoring for needle insertion: Effects of haptic and visual feedback on mentor perception of trainee forces,” in 2022 IEEE Haptics Symposium (HAPTICS), 2022, pp. 1-7.
S. Basu, J. Tsai, and A. Majewicz, “Evaluation of tactile guidance cue mappings for emergency percutaneous needle insertion,” in 2016 IEEE Haptics Symposium (HAPTICS), 2016, pp. 106-112.
C. Rossa, J. Fong, N. Usmani, R. Sloboda, and M. Tavakoli, “Multiactuator haptic feedback on the wrist for needle steering guidance in brachytherapy,” IEEE Robotics and Automation Letters, vol. 1, no. 2, pp. 852-859, 2016.
B. Weber, S. Schatzle, T. Hulin, C. Preusche, and B. Deml, “Evaluation” of a vibrotactile feedback device for spatial guidance,” in 2011 IEEE World Haptics Conference, 2011, pp. 349-354.
W. Guo, W. Ni, I.-M. Chen, Z. Ding, and S. Yeo, “Intuitive vibro-tactile feedback for human body movement guidance,” 01 2010, pp. 135-140.
M. Raitor, J. M. Walker, A. M. Okamura, and H. Culbertson, “WRAP: Wearable, restricted-aperture pneumatics for haptic guidance,” in 2017 IEEE International Conference on Robotics and Automation (ICRA), May 2017, pp. 427-432.
K. Bark, J. W. Wheeler, S. Premakumar, and M. R. Cutkosky, “Comparison of Skin Stretch and Vibrotactile Stimulation for Feedback of Proprioceptive Information,” in 2008 Symposium on Haptic Interfaces for Virtual Environment and Teleoperator Systems, March 2008, pp. 71-78, ISSN: 2324-7355.
K. Bark, J. Wheeler, P. Shull, J. Savall, and M. Cutkosky, “Rotational skin stretch feedback: a wearable haptic display for motion,” IEEE Transactions on Haptics, vol. 3, no. 3, pp. 166-176, 2010.
S. Casini, M. Morvidoni, M. Bianchi, M. Catalano, G. Grioli, and A. Bicchi, “Design and realization of the cuff—clenching upper limb force feedback wearable device for distributed mechano-tactile stimulation of normal and tangential skin forces,” in 2015 IEEE/RSJ International Conference on Intelligent Robots and Systems (IROS), 2015, pp. 1186-1193.
E. Pezent, S. Fani, J. Clark, M. Bianchi, and M. K. O'Malley, “Spatially Separating Haptic Guidance From Task Dynamics Through Wearable Devices,” IEEE Transactions on Haptics, vol. 12, no. 4, pp. 581-593, October 2019.
F. Chinello, C. Pacchierotti, J. Bimbo, N. G. Tsagarakis, and D. Prattichizzo, “Design and evaluation of a wearable skin stretch device for haptic guidance,” IEEE Robotics and Automation Letters, vol. 3, no. 1, pp. 524-531, 2018.
V. Yem, M. Otsuki, and H. Kuzuoka, Development of Wearable Outer Covering Haptic Display Using Ball Effector for Hand Motion Guidance. Tokyo: Springer Japan, 2015, pp. 85-89.
A. A. Stanley and K. J. Kuchenbecker, “Evaluation of Tactile Feedback Methods for Wrist Rotation Guidance,” IEEE Transactions on Haptics, vol. 5, no. 3, pp. 240-251, 2012.
A. Haynes, M. F. Simons, T. Helps, Y. Nakamura, and J. Rossiter, “A wearable skin-stretching tactile interface for human-robot and human-human communication,” IEEE Robotics and Automation Letters, vol. 4, no. 2, pp. 1641-1646, 2019.
B. Jumet, Z. A. Zook, A. Yousaf, A. Rajappan, D. Xu, T. F. Yap, N. Fino, Z. Liu, M. K. O'Malley, and D. J. Preston, “Fluidically programmed wearable haptic textiles,” Device, vol. 1, no. 3, p. 100059, 2023.
E. H. Skorina, M. Luo, and C. D. Onal, “A soft robotic wearable wrist device for kinesthetic haptic feedback,” Frontiers in Robotics and AI, vol. 5, 2018.
A. Erwin, M. K. O'Malley, D. Ress, and F. Sergi, “Kinesthetic feedback during 2dof wrist movements via a novel mr-compatible robot,” IEEE Transactions on Neural Systems and Rehabilitation Engineering, vol. 25, no. 9, pp. 1489-1499, 2017.
C.-Y. Chen, Y.-Y. Chen, Y.-J. Chung, and N.-H. Yu, “Motion guidance sleeve: Guiding the forearm rotation through external artificial muscles,” in Proceedings of the 2016 CHI Conference on Human Factors in Computing Systems, ser. CHI '16. New York, NY, USA: Association for Computing Machinery, 2016, p. 3272-3276.
Y. Kuniyasu, M. Sato, S. Fukushima, and H. Kajimoto, “Transmission of forearm motion by tangential deformation of the skin,” in Proceedings of the 3rd Augmented Human International Conference, ser. AH '12. New York, NY, USA: Association for Computing Machinery, 2012.
D. Margineanu, E.-C. Lovasz, C. M. Gruescu, V. Ciupe, and S. Tatar, “5 DoF Haptic Exoskeleton for Space Telerobotics—Shoulder Module,” in Mechanisms, Transmissions and Applications, ser. Mechanisms and Machine Science, M. I. C. Dede, M. Itik, E.-C. Lovasz, and G. Kiper, Eds. Cham: Springer International Publishing, 2018, pp. 111-120.
N. Agharese, T. Cloyd, L. H. Blumenschein, M. Raitor, E. W. Hawkes, H. Culbertson, and A. M. Okamura, “Hapwrap: Soft growing wearable haptic device,” in 2018 IEEE International Conference on Robotics and Automation (ICRA), 2018, pp. 5466-5472.
Y. Kuniyasu, S. Fukushima, M. Furukawa, and H. Kajimoto, “Weight illusion by tangential deformation of forearm skin,” in Proceedings of the 2nd Augmented Human International Conference, ser. AH '11. New York, NY, USA: Association for Computing Machinery, 2011.
J. M. Walker, H. Culbertson, M. Raitor, and A. M. Okamura, “Haptic orientation guidance using two parallel double-gimbal control moment gyroscopes,” IEEE Transactions on Haptics, vol. 11, no. 2, pp. 267-278, 2018.
ROS.org contributors, “rosserial: Ros client library for embedded devices,” https://github.com/ros-drivers/rosserial, 2024, accessed: 2025 Apr. 1.
K. N. Winfree, J. Gewirtz, T. Mather, J. Fiene, and K. J. Kuchenbecker, “A high fidelity ungrounded torque feedback device: The itorqu 2.0,” in World Haptics 2009—Third Joint EuroHaptics conference and Symposium on Haptic Interfaces for Virtual Environment and Teleoperator Systems, 2009, pp. 261-266.
M. Zhu, S. Biswas, S. I. Dinulescu, N. Kastor, E. W. Hawkes, and Y. Visell, “Soft, Wearable Robotics and Haptics: Technologies, Trends, and Emerging Applications,” Proceedings of the IEEE, vol. 110, no. 2, pp. 246-272, February 2022.
O. Kayhan, A. K. Nennioglu, and E. Samur, “A skin stretch tactor for sensory substitution of wrist proprioception,” in 2018 IEEE Haptics Symposium (HAPTICS), 2018, pp. 26-31.
H. Culbertson, S. B. Schorr, and A. M. Okamura, “Haptics: The present and future of artificial touch sensation,” Annual Review of Control, Robotics, and Autonomous Systems, vol. 1, no. 1, pp. 385-409, 2018.
A. Adilkhanov, M. Rubagotti, and Z. Kappassov, “Haptic devices: Wearability-based taxonomy and literature review,” IEEE Access, vol. 10, pp. 91923-91947, 2022.
M. Fleury, G. Lioi, C. Barillot, and A. Lecuyer, “A survey on the use' of haptic feedback for brain-computer interfaces and neurofeedback,” Frontiers in Neuroscience, vol. 14, 2020.
“Multi-functional textile and related methods of manufacturing,” Patent U.S. Pat. No. 11,077,655B2, Aug. 3, 2021.
P. Q. Lee, V. Rajendran, and K. Mombaur, “Optimization-based motion generation for buzzwire tasks with the reem-c humanoid robot,” Frontiers in Robotics and AI, vol. 9, 2022. [Online]. Available: https://www.frontiersin.org/articles/10.3389/frobt.2022.898890
B. Mathur, A. Topiwala, H. Saeidi, T. Fleiter, and A. Krieger, “Evaluation of control strategies for a tele-manipulated robotic system for remote trauma assessment,” in 2019 Proceedings of the Conference on Control and its Applications, 2019, pp. 7-14
U.S. Patent App. No. US20170119553, Entitled “A Haptic Feedback Device”
C. Pacchierotti, S. Sinclair, M. Solazzi, A. Frisoli, V. Hayward, and D. Prattichizzo, “Wearable haptic systems for the fingertip and the hand: taxonomy, review, and perspectives,” IEEE Transactions on Haptics, vol. 10, no. 4, pp. 580-600, 2017.
J. Fleck, Z. Zook, J. Clark et al., “Wearable multi-sensory haptic devices,” Nature Reviews Bioengineering, 2025.
K. T. Yoshida, Z. A. Zook, H. Choi, M. Luo, M. K. O'Malley, and A. M. Okamura, “Design and evaluation of a 3-dof haptic device for directional shear cues on the forearm,” IEEE Transactions on Haptics, vol. 17, no. 3, pp. 483-495, 2024.
A. R. See, J. A. G. Choco, and K. Chandramohan, “Touch, texture and haptic feedback: A review on how we feel the world around us,” Applied Sciences, vol. 12, no. 9, 2022.
T.-H. Yang, J. R. Kim, H. Jin, H. Gil, J.-H. Koo, and H. J. Kim, “Recent advances and opportunities of active materials for haptic technologies in virtual and augmented reality,” Advanced Functional Materials, vol. 31, no. 39, p. 2008831, 2021.
A. Alaraj, C. J. Luciano, D. P. Bailey, A. Elsenousi, B. Z. Roitberg, A. Bernardo, P. P. Banerjee, and F. T. Charbel, “Virtual reality cerebral aneurysm clipping simulation with real-time haptic feedback,” Operative Neurosurgery, vol. 11, no. 1, pp. 52-58, 2015.
M. Ershad, R. Rege, and A. M. Fey, “Adaptive surgical robotic training using real-time stylistic behavior feedback through haptic cues,” IEEE Transactions on Medical Robotics and Bionics, vol. 3, no. 4, pp. 959-969, 2021.
K. Hale, K. Stanney, and L. Malone, “Enhancing virtual environment spatial awareness training and transfer through tactile and vestibular cues,” Ergonomics, vol. 52, no. 2, pp. 187-203, 2009.
A. F. Galvan, J. D. Ramirez, A. D. Deshpande, and A. M. Fey, “An extended virtual proxy haptic algorithm for dexterous manipulation in virtual environments,” in 2023 IEEE World Haptics Conference (WHC). IEEE, 2023, pp. 176-182.
K. J. Kuchenbecker, J. Fiene, and G. Niemeyer, “Improving contact realism through event-based haptic feedback,” IEEE transactions on visualization and computer graphics, vol. 12, no. 2, pp. 219-230, 2006.
K. Bark, P. Khanna, R. Irwin, P. Kapur, and S. A. Jax, “Lessons in using vibrotactile feedback to guide fast arm motions,” in 2011 IEEE World Haptics Conference. IEEE, 2011, pp. 355-360.
H. Culbertson, J. M. Walker, M. Raitor, A. M. Okamura, and P. J. Stolka, “Plane assist: the influence of haptics on ultrasound-based needle guidance,” in Medical Image Computing and Computer-Assisted Intervention-MICCAI 2016. Springer, 2016, pp. 370-377.
E. Battaglia and A. M. Fey, “Cartesian Space Vibrotactile Cues Outperform Tool Space Cues when Moving from 2D to 3D Needle Insertion Task,” in 2022 9th IEEE RAS/EMBS International Conference for Biomedical Robotics and Biomechatronics (BioRob), August 2022, pp. 1-6, ISSN: 2155-1782.
L. R. Elliott, J. van Erp, E. S. Redden, and M. Duistermaat, “Field-based validation of a tactile navigation device,” IEEE Transactions on Haptics, vol. 3, no. 1, pp. 78-87, 2010.
A. Cosgun, E. A. Sisbot, and H. I. Christensen, “Guidance for human navigation using a vibro-tactile belt interface and robot-like motion planning,” in 2014 IEEE International Conference on Robotics and Automation (ICRA). IEEE, 2014, pp. 6350-6355.
J. Lee, B. J. Atwood, S. Megat, G. Dussor, T. J. Price, and A. M. Fey, “Haptic stroke testbed for pharmacological evaluation of dynamic allodynia in mouse models,” in 2018 IEEE Haptics Symposium (HAPTICS). IEEE, 2018, pp. 235-240.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A wearable haptic guidance and feedback device, comprising:
a sleeve;
a flexible grounding mechanism attached to the sleeve, configured to conform to a wearer's anatomy and distribute a reaction force to said anatomy;
at least one actuator attached to the grounding mechanism; and
at least one actuation mechanism mechanically coupling the at least one actuator to at least one of the sleeve and the grounding mechanism.

2. The device of claim 1, wherein the device is ungrounded.

3. The device of claim 1, further comprising one or more sensors.

4. The device of claim 3, wherein the one or more sensors comprise at least one of a flexible force sensor, an inertial measurement unit, a force sensor, a position sensor, a velocity sensor, an acceleration sensor, an orientation sensor, a flex sensor, and an encoder.

5. The device of claim 3, wherein the sleeve comprises at least two layers.

6. The device of claim 5, wherein wiring for the at least one actuator or sensor is between the layers of the sleeve.

7. The device of claim 1, wherein the grounding mechanism comprises chain mail.

8. The device of claim 7, wherein the chain mail comprises hexagons.

9. The device of claim 1, wherein the grounding mechanism is attached to the sleeve proximate to a bicep location and a forearm location.

10. The device of claim 1, wherein the grounding mechanism comprises a thread passing through at least a portion of the grounding mechanism, and a fixation mechanism configured to tighten the thread and grounding mechanism.

11. The device of claim 1, wherein the at least one actuator comprises eight actuators.

12. The device of claim 11, wherein two of the eight actuators are positioned in a first location of the grounding mechanism, and six of the eight actuators are positioned in a second location of the grounding mechanism.

13. The device of claim 12, wherein the first location is proximate to a forearm and the second location is proximate to a bicep.

14. The device of claim 1, where the at least one actuator comprises at least one of a servo motor, a brushed DC motor, a brushless DC motor, a linear actuator, an electroactive polymer, a shape memory alloy, and a pneumatic actuator.

15. The device of claim 1, wherein the at least one actuation mechanism comprises linear and tendon-driven actuation mechanisms.

16. The device of claim 15, wherein the linear actuation mechanisms comprise rack and pinion mechanisms operating flexible rods, a ball screw, a lead screw, or a pneumatic, and wherein the tendon-driven actuation mechanisms comprise a tendon comprising a cord.

17. The device of claim 16, further comprising actuated anchoring points interfacing with at least one of the rods or tendons.

18. The device of claim 16, wherein the rods and tendons comprise a plastic or a metal.

19. A wearable haptic guidance and feedback system, comprising:
the wearable haptic guidance and feedback device of claim 1; and
a computing system communicatively connected to the wearable haptic guidance and feedback device.

20. The system of claim 19, wherein the computing system comprises a processor and a non-transitory computer-readable medium with instructions stored thereon, which when executed by the processor, perform steps comprising:
actuating the at least one actuator.
